SEQUENCE LISTING (19 sequences)

SEQ ID NO: 1 - protein, 34 aa, Artificial Sequence
  Synthetic Construct
  MISC_FEATURE (2)..(2): Xaa at position 2 is Aib
  MOD_RES (20)..(20): Lys at position 20 is chemically modified by conjugation of the epsilon-amino group of the Lys side chain with ([2-(2-aminoethoxy)-ethoxy]-acetyl)2-(gamma-Glu)-CO-(CH2)18CO2H
  MOD_RES (34)..(34): Gly at position 34 is amidated
HXQGTFTSDYSKYLDEKKAKEFVEWLLEGGPSSG

SEQ ID NO: 2 - protein, 34 aa, Artificial Sequence; identical to SEQ ID NO: 1
  Synthetic Construct
  MOD_RES (1)..(1): N-terminal Histidine is protected by the protecting group PG1, wherein PG1 is a base stable side-chain protecting group
  MOD_RES (1)..(1): Sidechain of Histidine is protected by the protecting group PG1, wherein PG1 is a base stable side-chain protecting group
  MISC_FEATURE (2)..(2): Xaa at position 2 is Aib
  MOD_RES (3)..(3): Sidechain of Glutamine is protected by the protecting group PG1, wherein PG1 is a base stable side-chain protecting group
  MOD_RES (5)..(5): Sidechain of Threonine is optionally protected by the protecting group PG1, wherein PG1 is a base stable side-chain protecting group
  MOD_RES (7)..(7): Sidechain of Threonine is protected by the protecting group PG1, wherein PG1 is a base stable side-chain protecting group
  MOD_RES (8)..(8): Sidechain of Serine is protected by the protecting group PG1, wherein PG1 is a base stable side-chain protecting group
  MOD_RES (9)..(9): Sidechain of Aspartic acid is protected by the protecting group PG1, wherein PG1 is a base stable side-chain protecting group
  MOD_RES (10)..(10): Sidechain of Tyrosine is protected by the protecting group PG1, wherein PG1 is a base stable side-chain protecting group
  MOD_RES (11)..(11): Sidechain of Serine is protected by the protecting group PG1, wherein PG1 is a base stable side-chain protecting group
  MOD_RES (12)..(12): Sidechain of Lysine is protected by the protecting group PG1, wherein PG1 is a base stable side-chain protecting group
  MOD_RES (13)..(13): Sidechain of Tyrosine is protected by the protecting group PG1, wherein PG1 is a base stable side-chain protecting group
  MOD_RES (15)..(15): Sidechain of Aspartic acid is protected by the protecting group PG1, wherein PG1 is a base stable side-chain protecting group
  MOD_RES (16)..(16): Sidechain of Glutamic acid is protected by the protecting group PG1, wherein PG1 is a base stable side-chain protecting group
  MOD_RES (17)..(17): Sidechain of Lysine is protected by the protecting group PG1, wherein PG1 is a base stable side-chain protecting group
  MOD_RES (18)..(18): Sidechain of Lysine is protected by the protecting group PG1, wherein PG1 is a base stable side-chain protecting group
  MOD_RES (20)..(20): Sidechain of Lysine is protected by the protecting group PG2, wherein PG2 is an ivDde, Dde or Alloc side-chain protecting group
  MOD_RES (21)..(21): Sidechain of Glutamic acid is protected by the protecting group PG1, wherein PG1 is a base stable side-chain protecting group
  MOD_RES (24)..(24): Sidechain of Glutamic acid is protected by the protecting group PG1, wherein PG1 is a base stable side-chain protecting group
  MOD_RES (25)..(25): Sidechain of Tryptophan is protected by the protecting group PG1, wherein PG1 is a base stable side-chain protecting group
  MOD_RES (28)..(28): Sidechain of Glutamic acid is protected by the protecting group PG1, wherein PG1 is a base stable side-chain protecting group
  MOD_RES (32)..(32): Sidechain of Serine is protected by the protecting group PG1, wherein PG1 is a base stable side-chain protecting group
  MOD_RES (33)..(33): Sidechain of Serine is protected by the protecting group PG1, wherein PG1 is a base stable side-chain protecting group
  MOD_RES (34)..(34): The C-terminal Glycine is amidated and is bound to Sieber resin through this amine group

SEQ ID NO: 3 - protein, 34 aa, Artificial Sequence; identical to SEQ ID NO: 1
  Synthetic Construct
  MOD_RES (1)..(1): N-terminal Histidine is protected by Boc
  MOD_RES (1)..(1): Sidechain of Histidine is protected by Boc
  MISC_FEATURE (2)..(2): Xaa at position 2 is Aib
  MOD_RES (3)..(3): Sidechain of Glutamine is protected by Trityl
  MOD_RES (7)..(7): Sidechain of Threonine is protected by t-Butyl
  MOD_RES (8)..(8): Sidechain of Serine is protected by t-Butyl
  MOD_RES (9)..(9): Sidechain of Aspartic acid is protected by t-Butyl
  MOD_RES (10)..(10): Sidechain of Tyrosine is protected by t-Butyl
  MOD_RES (11)..(11): Sidechain of Serine is protected by t-Butyl
  MOD_RES (12)..(12): Sidechain of Lysine is protected by Boc
  MOD_RES (13)..(13): Sidechain of Tyrosine is protected by t-Butyl
  MOD_RES (15)..(15): Sidechain of Aspartic acid is protected by t-Butyl
  MOD_RES (16)..(16): Sidechain of Glutamic acid is protected by t-Butyl
  MOD_RES (17)..(17): Sidechain of Lysine is protected by Boc
  MOD_RES (18)..(18): Sidechain of Lysine is protected by Boc
  MOD_RES (20)..(20): Sidechain of Lysine is protected by ivDde
  MOD_RES (21)..(21): Sidechain of Glutamic acid is protected by t-Butyl
  MOD_RES (24)..(24): Sidechain of Glutamic acid is protected by t-Butyl
  MOD_RES (25)..(25): Sidechain of Tryptophan is protected by Boc
  MOD_RES (28)..(28): Sidechain of Glutamic acid is protected by t-Butyl
  MOD_RES (32)..(32): Sidechain of Serine is protected by t-Butyl
  MOD_RES (33)..(33): Sidechain of Serine is protected by t-Butyl
  MOD_RES (34)..(34): The C-terminal Glycine is amidated and is bound to Sieber resin through this amine group

SEQ ID NO: 4 - protein, 34 aa, Artificial Sequence; identical to SEQ ID NO: 1
  Synthetic Construct
  MOD_RES (1)..(1): N-terminal Histidine is protected by Boc
  MOD_RES (1)..(1): Sidechain of Histidine is protected by Dnp
  MISC_FEATURE (2)..(2): Xaa at position 2 is Aib
  MOD_RES (3)..(3): Sidechain of Glutamine is protected by Trityl
  MOD_RES (5)..(5): Sidechain of Threonine is protected by t-Butyl
  MOD_RES (7)..(7): Sidechain of Threonine is protected by t-Butyl
  MOD_RES (8)..(8): Sidechain of Serine is protected by t-Butyl
  MOD_RES (9)..(9): Sidechain of Aspartic acid is protected by t-Butyl
  MOD_RES (10)..(10): Sidechain of Tyrosine is protected by t-Butyl
  MOD_RES (11)..(11): Sidechain of Serine is protected by t-Butyl
  MOD_RES (12)..(12): Sidechain of Lysine is protected by Boc
  MOD_RES (13)..(13): Sidechain of Tyrosine is protected by t-Butyl
  MOD_RES (15)..(15): Sidechain of Aspartic acid is protected by t-Butyl
  MOD_RES (16)..(16): Sidechain of Glutamic acid is protected by t-Butyl
  MOD_RES (17)..(17): Sidechain of Lysine is protected by Boc
  MOD_RES (18)..(18): Sidechain of Lysine is protected by Boc
  MOD_RES (20)..(20): Sidechain of Lysine is protected by ivDde
  MOD_RES (21)..(21): Sidechain of Glutamic acid is protected by t-Butyl
  MOD_RES (24)..(24): Sidechain of Glutamic acid is protected by t-Butyl
  MOD_RES (25)..(25): Sidechain of Tryptophan is protected by Boc
  MOD_RES (28)..(28): Sidechain of Glutamic acid is protected by t-Butyl
  MOD_RES (32)..(32): Sidechain of Serine is protected by t-Butyl
  MOD_RES (33)..(33): Sidechain of Serine is protected by t-Butyl
  MOD_RES (34)..(34): The C-terminal Glycine is amidated and is bound to Sieber resin through this amine group

SEQ ID NO: 5 - protein, 34 aa, Artificial Sequence; identical to SEQ ID NO: 1
  Synthetic Construct
  MOD_RES (1)..(1): N-terminal Histidine is protected by the protecting group PG1, wherein PG1 is a base stable side-chain protecting group
  MOD_RES (1)..(1): Sidechain of Histidine is protected by the protecting group PG1, wherein PG1 is a base stable side-chain protecting group
  MISC_FEATURE (2)..(2): Xaa at position 2 is Aib
  MOD_RES (3)..(3): Sidechain of Glutamine is protected by the protecting group PG1, wherein PG1 is a base stable side-chain protecting group
  MOD_RES (5)..(5): Sidechain of Threonine is optionally protected by the protecting group PG1, wherein PG1 is a base stable side-chain protecting group
  MOD_RES (7)..(7): Sidechain of Threonine is protected by the protecting group PG1, wherein PG1 is a base stable side-chain protecting group
  MOD_RES (8)..(8): Sidechain of Serine is protected by the protecting group PG1, wherein PG1 is a base stable side-chain protecting group
  MOD_RES (9)..(9): Sidechain of Aspartic acid is protected by the protecting group PG1, wherein PG1 is a base stable side-chain protecting group
  MOD_RES (10)..(10): Sidechain of Tyrosine is protected by the protecting group PG1, wherein PG1 is a base stable side-chain protecting group
  MOD_RES (11)..(11): Sidechain of Serine is protected by the protecting group PG1, wherein PG1 is a base stable side-chain protecting group
  MOD_RES (12)..(12): Sidechain of Lysine is protected by the protecting group PG1, wherein PG1 is a base stable side-chain protecting group
  MOD_RES (13)..(13): Sidechain of Tyrosine is protected by the protecting group PG1, wherein PG1 is a base stable side-chain protecting group
  MOD_RES (15)..(15): Sidechain of Aspartic acid is protected by the protecting group PG1, wherein PG1 is a base stable side-chain protecting group
  MOD_RES (16)..(16): Sidechain of Glutamic acid is protected by the protecting group PG1, wherein PG1 is a base stable side-chain protecting group
  MOD_RES (17)..(17): Sidechain of Lysine is protected by the protecting group PG1, wherein PG1 is a base stable side-chain protecting group
  MOD_RES (18)..(18): Sidechain of Lysine is protected by the protecting group PG1, wherein PG1 is a base stable side-chain protecting group
  MOD_RES (21)..(21): Sidechain of Glutamic acid is protected by the protecting group PG1, wherein PG1 is a base stable side-chain protecting group
  MOD_RES (24)..(24): Sidechain of Glutamic acid is protected by the protecting group PG1, wherein PG1 is a base stable side-chain protecting group
  MOD_RES (25)..(25): Sidechain of Tryptophan is protected by the protecting group PG1, wherein PG1 is a base stable side-chain protecting group
  MOD_RES (28)..(28): Sidechain of Glutamic acid is protected by the protecting group PG1, wherein PG1 is a base stable side-chain protecting group
  MOD_RES (32)..(32): Sidechain of Serine is protected by the protecting group PG1, wherein PG1 is a base stable side-chain protecting group
  MOD_RES (33)..(33): Sidechain of Serine is protected by the protecting group PG1, wherein PG1 is a base stable side-chain protecting group
  MOD_RES (34)..(34): The C-terminal Glycine is amidated and is bound to Sieber resin through this amine group

SEQ ID NO: 6 - protein, 34 aa, Artificial Sequence; identical to SEQ ID NO: 1
  Synthetic Construct
  MOD_RES (1)..(1): N-terminal Histidine is protected by Boc
  MOD_RES (1)..(1): Sidechain of Histidine is protected by Boc
  MISC_FEATURE (2)..(2): Xaa at position 2 is Aib
  MOD_RES (3)..(3): The sidechain of Glutamine is protected by Trityl
  MOD_RES (7)..(7): The sidechain of Threonine is protected by t-Butyl
  MOD_RES (8)..(8): The sidechain of Serine is protected by t-Butyl
  MOD_RES (9)..(9): The sidechain of Aspartic acid is protected by t-Butyl
  MOD_RES (10)..(10): The sidechain of Tyrosine is protected by t-Butyl
  MOD_RES (11)..(11): The sidechain of Serine is protected by t-Butyl
  MOD_RES (12)..(12): The sidechain of Lysine is protected by Boc
  MOD_RES (13)..(13): The sidechain of Tyrosine is protected by t-Butyl
  MOD_RES (15)..(15): The sidechain of Aspartic acid is protected by t-Butyl
  MOD_RES (16)..(16): The sidechain of Glutamic acid is protected by t-Butyl
  MOD_RES (17)..(17): The sidechain of Lysine is protected by Boc
  MOD_RES (18)..(18): The sidechain of Lysine is protected by Boc
  MOD_RES (21)..(21): The sidechain of Glutamic acid is protected by t-Butyl
  MOD_RES (24)..(24): The sidechain of Glutamic acid is protected by t-Butyl
  MOD_RES (25)..(25): The sidechain of Tryptophan is protected by Boc
  MOD_RES (28)..(28): The sidechain of Glutamic acid is protected by t-Butyl
  MOD_RES (32)..(32): The sidechain of Serine is protected by t-Butyl
  MOD_RES (33)..(33): The sidechain of Serine is protected by t-Butyl
  MOD_RES (34)..(34): The C-terminal Glycine is amidated and is bound to Sieber resin through this amine group

SEQ ID NO: 7 - protein, 34 aa, Artificial Sequence; identical to SEQ ID NO: 1
  Synthetic Construct
  MOD_RES (1)..(1): N-terminal Histidine is protected by the protecting group PG1, wherein PG1 is a base stable side-chain protecting group
  MOD_RES (1)..(1): Sidechain of Histidine is protected by the protecting group PG1, wherein PG1 is a base stable side-chain protecting group
  MISC_FEATURE (2)..(2): Xaa at position 2 is Aib
  MOD_RES (3)..(3): Sidechain of Glutamine is protected by the protecting group PG1, wherein PG1 is a base stable side-chain protecting group
  MOD_RES (5)..(5): Sidechain of Threonine is optionally protected by the protecting group PG1, wherein PG1 is a base stable side-chain protecting group
  MOD_RES (7)..(7): Sidechain of Threonine is protected by the protecting group PG1, wherein PG1 is a base stable side-chain protecting group
  MOD_RES (8)..(8): Sidechain of Serine is protected by the protecting group PG1, wherein PG1 is a base stable side-chain protecting group
  MOD_RES (9)..(9): Sidechain of Aspartic acid is protected by the protecting group PG1, wherein PG1 is a base stable side-chain protecting group
  MOD_RES (10)..(10): Sidechain of Tyrosine is protected by the protecting group PG1, wherein PG1 is a base stable side-chain protecting group
  MOD_RES (11)..(11): Sidechain of Serine is protected by the protecting group PG1, wherein PG1 is a base stable side-chain protecting group
  MOD_RES (12)..(12): Sidechain of Lysine is protected by the protecting group PG1, wherein PG1 is a base stable side-chain protecting group
  MOD_RES (13)..(13): Sidechain of Tyrosine is protected by the protecting group PG1, wherein PG1 is a base stable side-chain protecting group
  MOD_RES (15)..(15): Sidechain of Aspartic acid is protected by the protecting group PG1, wherein PG1 is a base stable side-chain protecting group
  MOD_RES (16)..(16): Sidechain of Glutamic acid is protected by the protecting group PG1, wherein PG1 is a base stable side-chain protecting group
  MOD_RES (17)..(17): Sidechain of Lysine is protected by the protecting group PG1, wherein PG1 is a base stable side-chain protecting group
  MOD_RES (18)..(18): Sidechain of Lysine is protected by the protecting group PG1, wherein PG1 is a base stable side-chain protecting group
  MOD_RES (20)..(20): Lysine at position 20 is chemically modified through conjugation to the epsilon-amino group of the K side- chain with (2-[2-(2-Amino-ethoxy)-ethoxy]-acetyl)2-(gamma- Glu(t-butyl))-CO-(CH2)18-CO2-(t-butyl)
  MOD_RES (21)..(21): Sidechain of Glutamic acid is protected by the protecting group PG1, wherein PG1 is a base stable side-chain protecting group
  MOD_RES (24)..(24): Sidechain of Glutamic acid is protected by the protecting group PG1, wherein PG1 is a base stable side-chain protecting group
  MOD_RES (25)..(25): Sidechain of Tryptophan is protected by the protecting group PG1, wherein PG1 is a base stable side-chain protecting group
  MOD_RES (28)..(28): Sidechain of Glutamic acid is protected by the protecting group PG1, wherein PG1 is a base stable side-chain protecting group
  MOD_RES (32)..(32): Sidechain of Serine is protected by the protecting group PG1, wherein PG1 is a base stable side-chain protecting group
  MOD_RES (33)..(33): Sidechain of Serine is protected by the protecting group PG1, wherein PG1 is a base stable side-chain protecting group
  MOD_RES (34)..(34): The C-terminal Glycine is amidated and is bound to Sieber resin through this amine group

SEQ ID NO: 8 - protein, 34 aa, Artificial Sequence; identical to SEQ ID NO: 1
  Synthetic Construct
  MOD_RES (1)..(1): N-terminal Histidine is protected by Boc
  MOD_RES (1)..(1): Sidechain of Histidine is protected by Boc
  MISC_FEATURE (2)..(2): Xaa at position 2 is Aib
  MOD_RES (3)..(3): Sidechain of Glutamine is protected by Trityl
  MOD_RES (7)..(7): Sidechain of Threonine is protected by t-Butyl
  MOD_RES (8)..(8): Sidechain of Serine is protected by t-Butyl
  MOD_RES (9)..(9): Sidechain of Aspartic acid is protected by t-Butyl
  MOD_RES (10)..(10): Sidechain of Tyrosine is protected by t-Butyl
  MOD_RES (11)..(11): Sidechain of Serine is protected by t-Butyl
  MOD_RES (12)..(12): Sidechain of Lysine is protected by Boc
  MOD_RES (13)..(13): Sidechain of Tyrosine is protected by t-Butyl
  MOD_RES (15)..(15): Sidechain of Aspartic acid is protected by t-Butyl
  MOD_RES (16)..(16): Sidechain of Glutamic acid is protected by t-Butyl
  MOD_RES (17)..(17): Sidechain of Lysine is protected by Boc
  MOD_RES (18)..(18): Sidechain of Lysine is protected by Boc
  MOD_RES (20)..(20): Lysine at position 20 is chemically modified through conjugation to the epsilon-amino group of the K side- chain with (2-[2-(2-Amino-ethoxy)-ethoxy]-acetyl)2-(gamma- Glu(t-butyl))-CO-(CH2)18-CO2-(t-butyl)
  MOD_RES (21)..(21): Sidechain of Glutamic acid is protected by t-Butyl
  MOD_RES (24)..(24): Sidechain of Glutamic acid is protected by t-Butyl
  MOD_RES (25)..(25): Sidechain of Tryptophan is protected by Boc
  MOD_RES (28)..(28): Sidechain of Glutamic acid is protected by t-Butyl
  MOD_RES (32)..(32): Sidechain of Serine is protected by t-Butyl
  MOD_RES (33)..(33): Sidechain of Serine is protected by t-Butyl
  MOD_RES (34)..(34): The C-terminal Glycine is amidated and is bound to Sieber resin through this amine group

SEQ ID NO: 9 - protein, 29 aa, Artificial Sequence
  Synthetic Construct
  MOD_RES (1)..(1): N-terminal Phenylalanine is protected by the protecting group PG1, wherein PG1 is a base stable side-chain protecting group
  MOD_RES (2)..(2): Sidechain of Threonine is protected by the protecting group PG1, wherein PG1 is a base stable side-chain protecting group
  MOD_RES (3)..(3): Sidechain of Serine is protected by the protecting group PG1, wherein PG1 is a base stable side-chain protecting group
  MOD_RES (4)..(4): Sidechain of Aspartic acid is protected by the protecting group PG1, wherein PG1 is a base stable side-chain protecting group
  MOD_RES (5)..(5): Sidechain of Tyrosine is protected by the protecting group PG1, wherein PG1 is a base stable side-chain protecting group
  MOD_RES (6)..(6): Sidechain of Serine is protected by the protecting group PG1, wherein PG1 is a base stable side-chain protecting group
  MOD_RES (7)..(7): Sidechain of Lysine is protected by the protecting group PG1, wherein PG1 is a base stable side-chain protecting group
  MOD_RES (8)..(8): Sidechain of Tyrosine is protected by the protecting group PG1, wherein PG1 is a base stable side-chain protecting group
  MOD_RES (10)..(10): Sidechain of Aspartic acid is protected by the protecting group PG1, wherein PG1 is a base stable side-chain protecting group
  MOD_RES (11)..(11): Sidechain of Glutamic acid is protected by the protecting group PG1, wherein PG1 is a base stable side-chain protecting group
  MOD_RES (12)..(12): Sidechain of Lysine is protected by the protecting group PG1, wherein PG1 is a base stable side-chain protecting group
  MOD_RES (13)..(13): Sidechain of Lysine is protected by the protecting group PG1, wherein PG1 is a base stable side-chain protecting group
  MOD_RES (15)..(15): Sidechain of Lysine is protected by the protecting group PG2, wherein PG2 is an ivDde, Dde or Alloc side-chain protecting group
  MOD_RES (16)..(16): Sidechain of Glutamic acid is protected by the protecting group PG1, wherein PG1 is a base stable side-chain protecting group
  MOD_RES (19)..(19): Sidechain of Glutamic acid is protected by the protecting group PG1, wherein PG1 is a base stable side-chain protecting group
  MOD_RES (20)..(20): Sidechain of Tryptophan is protected by the protecting group PG1, wherein PG1 is a base stable side-chain protecting group
  MOD_RES (23)..(23): Sidechain of Glutamic acid is protected by the protecting group PG1, wherein PG1 is a base stable side-chain protecting group
  MOD_RES (27)..(27): Sidechain of Serine is protected by the protecting group PG1, wherein PG1 is a base stable side-chain protecting group
  MOD_RES (28)..(28): Sidechain of Serine is protected by the protecting group PG1, wherein PG1 is a base stable side-chain protecting group
  MOD_RES (29)..(29): The C-terminal Glycine is amidated and is bound to resin through this amine group
FTSDYSKYLDEKKAKEFVEWLLEGGPSSG

SEQ ID NO: 10 - protein, 29 aa, Artificial Sequence
  Synthetic Construct
  MOD_RES (1)..(1): N-terminal Phenylalanine is protected by Fmoc
  MOD_RES (2)..(2): Sidechain of Threonine is protected by t-Butyl
  MOD_RES (3)..(3): Sidechain of Serine is protected by t-Butyl
  MOD_RES (4)..(4): Sidechain of Aspartic acid is protected by t-Butyl
  MOD_RES (5)..(5): Sidechain of Tyrosine is protected by t-Butyl
  MOD_RES (6)..(6): Sidechain of Serine is protected by t-Butyl
  MOD_RES (7)..(7): Sidechain of Lysine is protected by Boc
  MOD_RES (8)..(8): Sidechain of Tyrosine is protected by t-Butyl
  MOD_RES (10)..(10): Sidechain of Aspartic acid is protected by t-Butyl
  MOD_RES (11)..(11): Sidechain of Glutamic acid is protected by t-Butyl
  MOD_RES (12)..(12): Sidechain of Lysine is protected by Boc
  MOD_RES (13)..(13): Sidechain of Lysine is protected by Boc
  MOD_RES (15)..(15): Sidechain of Lysine is protected by ivDde
  MOD_RES (16)..(16): Sidechain of Glutamic acid is protected by t-Butyl
  MOD_RES (19)..(19): Sidechain of Glutamic acid is protected by t-Butyl
  MOD_RES (20)..(20): Sidechain of Tryptophan is protected by Boc
  MOD_RES (23)..(23): Sidechain of Glutamic acid is protected by t-Butyl
  MOD_RES (27)..(27): Sidechain of Serine is protected by t-Butyl
  MOD_RES (28)..(28): Sidechain of Serine is protected by t-Butyl
  MOD_RES (29)..(29): The C-terminal Glycine is amidated and is bound to Sieber resin through this amine group
FTSDYSKYLDEKKAKEFVEWLLEGGPSSG

SEQ ID NO: 11 - protein, 30 aa, Artificial Sequence
  Synthetic Construct
  MOD_RES (1)..(1): N-terminal Threonine is protected by the protecting group PG1, wherein PG1 is a base stable side-chain protecting group
  MOD_RES (1)..(1): Sidechain of Threonine is protected by the protecting group PG1, wherein PG1 is a base stable side-chain protecting group
  MOD_RES (3)..(3): Sidechain of Threonine is protected by the protecting group PG1, wherein PG1 is a base stable side-chain protecting group
  MOD_RES (4)..(4): Sidechain of Serine is protected by the protecting group PG1, wherein PG1 is a base stable side-chain protecting group
  MOD_RES (5)..(5): Sidechain of Aspartic acid is protected by the protecting group PG1, wherein PG1 is a base stable side-chain protecting group
  MOD_RES (6)..(6): Sidechain of Tyrosine is protected by the protecting group PG1, wherein PG1 is a base stable side-chain protecting group
  MOD_RES (7)..(7): Sidechain of Serine is protected by the protecting group PG1, wherein PG1 is a base stable side-chain protecting group
  MOD_RES (8)..(8): Sidechain of Lysine is protected by the protecting group PG1, wherein PG1 is a base stable side-chain protecting group
  MOD_RES (9)..(9): Sidechain of Tyrosine is protected by the protecting group PG1, wherein PG1 is a base stable side-chain protecting group
  MOD_RES (11)..(11): Sidechain of Aspartic acid is protected by the protecting group PG1, wherein PG1 is a base stable side-chain protecting group
  MOD_RES (12)..(12): Sidechain of Glutamic acid is protected by the protecting group PG1, wherein PG1 is a base stable side-chain protecting group
  MOD_RES (13)..(13): Sidechain of Lysine is protected by the protecting group PG1, wherein PG1 is a base stable side-chain protecting group
  MOD_RES (14)..(14): Sidechain of Lysine is protected by the protecting group PG1, wherein PG1 is a base stable side-chain protecting group
  MOD_RES (16)..(16): Sidechain of Lysine is protected by the protecting group PG2, wherein PG2 is an ivDde, Dde or Alloc side-chain protecting group
  MOD_RES (17)..(17): Sidechain of Glutamic acid is protected by the protecting group PG1, wherein PG1 is a base stable side-chain protecting group
  MOD_RES (20)..(20): Sidechain of Glutamic acid is protected by the protecting group PG1, wherein PG1 is a base stable side-chain protecting group
  MOD_RES (21)..(21): Sidechain of Tryptophan is protected by the protecting group PG1, wherein PG1 is a base stable side-chain protecting group
  MOD_RES (24)..(24): Sidechain of Glutamic acid is protected by the protecting group PG1, wherein PG1 is a base stable side-chain protecting group
  MOD_RES (28)..(28): Sidechain of Serine is protected by the protecting group PG1, wherein PG1 is a base stable side-chain protecting group
  MOD_RES (29)..(29): Sidechain of Serine is protected by the protecting group PG1, wherein PG1 is a base stable side-chain protecting group
  MOD_RES (29)..(29): The C-terminal Glycine is amidated and is bound to Sieber resin through this amine group
TFTSDYSKYLDEKKAKEFVEWLLEGGPSSG

SEQ ID NO: 12 - protein, 30 aa, Artificial Sequence; identical to SEQ ID NO: 11
  Synthetic Construct
  MOD_RES (1)..(1): N-terminal Threonine is protected by Fmoc
  MOD_RES (1)..(1): Sidechain of Threonine is protected by t-Butyl
  MOD_RES (3)..(3): Sidechain of Threonine is protected by t-Butyl
  MOD_RES (4)..(4): Sidechain of Serine is protected by t-Butyl
  MOD_RES (5)..(5): Sidechain of Aspartic acid is protected by t-Butyl
  MOD_RES (6)..(6): Sidechain of Tyrosine is protected by t-Butyl
  MOD_RES (7)..(7): Sidechain of Serine is protected by t-Butyl
  MOD_RES (8)..(8): Sidechain of Lysine is protected by Boc
  MOD_RES (9)..(9): Sidechain of Tyrosine is protected by t-Butyl
  MOD_RES (11)..(11): Sidechain of Aspartic acid is protected by t-Butyl
  MOD_RES (12)..(12): Sidechain of Glutamic acid is protected by t-Butyl
  MOD_RES (13)..(13): Sidechain of Lysine is protected by Boc
  MOD_RES (14)..(14): Sidechain of Lysine is protected by Boc
  MOD_RES (16)..(16): Sidechain of Lysine is protected by ivDde
  MOD_RES (17)..(17): Sidechain of Glutamic acid is protected by t-Butyl
  MOD_RES (20)..(20): Sidechain of Glutamic acid is protected by t-Butyl
  MOD_RES (21)..(21): Sidechain of Tryptophan is protected by Boc
  MOD_RES (24)..(24): Sidechain of Glutamic acid is protected by t-Butyl
  MOD_RES (28)..(28): Sidechain of Serine is protected by t-Butyl
  MOD_RES (29)..(29): Sidechain of Serine is protected by t-Butyl
  MOD_RES (30)..(30): The C-terminal Glycine is amidated and is bound to Sieber resin through this amine group

SEQ ID NO: 13 - protein, 5 aa, Artificial Sequence
  Synthetic Construct
  MOD_RES (1)..(1): N-terminal Histidine is protected by the protecting group PG1, wherein PG1 is a base stable side-chain protecting group
  MOD_RES (1)..(1): Sidechain of Histidine is protected by the protecting group PG1, wherein PG1 is a base stable side-chain protecting group
  MISC_FEATURE (2)..(2): Xaa at position 2 is Aib
  MOD_RES (3)..(3): Sidechain of Glutamine is protected by the protecting group PG1, wherein PG1 is a base stable side-chain protecting group
  MOD_RES (5)..(5): Sidechain of Threonine is protected by the protecting group PG1, wherein PG1 is a base stable side-chain protecting group
HXQGT

SEQ ID NO: 14 - protein, 5 aa, Artificial Sequence
  Synthetic Construct
  MOD_RES (1)..(1): N-terminal Histidine is protected by Boc
  MOD_RES (1)..(1): Sidechain of Histidine is protected by Dnp
  MISC_FEATURE (2)..(2): Xaa at position 2 is Aib
  MOD_RES (3)..(3): Sidechain of Glutamine is protected by Trityl
  MOD_RES (5)..(5): Sidechain of Threonine is protected by t-Butyl
HXQGT

SEQ ID NO: 15 - protein, 4 aa, Artificial Sequence
  Synthetic Construct
  MOD_RES (1)..(1): N-terminal Histidine is protected by the protecting group PG1, wherein PG1 is a base stable side-chain protecting group
  MOD_RES (1)..(1): Sidechain of Histidine is protected by the protecting group PG1, wherein PG1 is a base stable side-chain protecting group
  MISC_FEATURE (2)..(2): Xaa at position 2 is Aib
  MOD_RES (3)..(3): Sidechain of Glutamine is protected by the protecting group PG1, wherein PG1 is a base stable side-chain protecting group
HXQG

SEQ ID NO: 16 - protein, 4 aa, Artificial Sequence
  Synthetic Construct
  MOD_RES (1)..(1): N-terminal Histidine is protected by Boc
  MOD_RES (1)..(1): Sidechain of Histidine is protected by Dnp
  MISC_FEATURE (2)..(2): Xaa at position 2 is Aib
  MOD_RES (3)..(3): Sidechain of Glutamine is protected by Trityl
HXQG

SEQ ID NO: 17 - protein, 34 aa, Artificial Sequence; identical to SEQ ID NO: 1
  Synthetic Construct
  MOD_RES (1)..(1): N-terminal Histidine is protected by the protecting group PG1, wherein PG1 is a base stable side-chain protecting group
  MOD_RES (1)..(1): Sidechain of Histidine is protected by the protecting group PG1, wherein PG1 is a base stable side-chain protecting group
  MISC_FEATURE (2)..(2): Xaa at position 2 is Aib
  MOD_RES (3)..(3): Sidechain of Glutamine is protected by the protecting group PG1, wherein PG1 is a base stable side-chain protecting group
  MOD_RES (5)..(5): Sidechain of Threonine is protected by the protecting group PG1, wherein PG1 is a base stable side-chain protecting group
  MOD_RES (7)..(7): Sidechain of Threonine is protected by the protecting group PG1, wherein PG1 is a base stable side-chain protecting group
  MOD_RES (8)..(8): Sidechain of Serine is protected by the protecting group PG1, wherein PG1 is a base stable side-chain protecting group
  MOD_RES (9)..(9): Sidechain of Aspartic acid is protected by the protecting group PG1, wherein PG1 is a base stable side-chain protecting group
  MOD_RES (10)..(10): Sidechain of Tyrosine is protected by the protecting group PG1, wherein PG1 is a base stable side-chain protecting group
  MOD_RES (11)..(11): Sidechain of Serine is protected by the protecting group PG1, wherein PG1 is a base stable side-chain protecting group
  MOD_RES (12)..(12): Sidechain of Lysine is protected by the protecting group PG1, wherein PG1 is a base stable side-chain protecting group
  MOD_RES (13)..(13): Sidechain of Tyrosine is protected by the protecting group PG1, wherein PG1 is a base stable side-chain protecting group
  MOD_RES (15)..(15): Sidechain of Aspartic acid is protected by the protecting group PG1, wherein PG1 is a base stable side-chain protecting group
  MOD_RES (16)..(16): Sidechain of Glutamic acid is protected by the protecting group PG1, wherein PG1 is a base stable side-chain protecting group
  MOD_RES (17)..(17): Sidechain of Lysine is protected by the protecting group PG1, wherein PG1 is a base stable side-chain protecting group
  MOD_RES (18)..(18): Sidechain of Lysine is protected by the protecting group PG1, wherein PG1 is a base stable side-chain protecting group
  MOD_RES (20)..(20): Sidechain of Lysine is protected by the protecting group PG2, wherein PG2 is an ivDde, Dde or Alloc side-chain protecting group
  MOD_RES (21)..(21): Sidechain of Glutamic acid is protected by the protecting group PG1, wherein PG1 is a base stable side-chain protecting group
  MOD_RES (24)..(24): Sidechain of Glutamic acid is protected by the protecting group PG1, wherein PG1 is a base stable side-chain protecting group
  MOD_RES (25)..(25): Sidechain of Tryptophan is protected by the protecting group PG1, wherein PG1 is a base stable side-chain protecting group
  MOD_RES (28)..(28): Sidechain of Glutamic acid is protected by the protecting group PG1, wherein PG1 is a base stable side-chain protecting group
  MOD_RES (32)..(32): Sidechain of Serine is protected by the protecting group PG1, wherein PG1 is a base stable side-chain protecting group
  MOD_RES (33)..(33): Sidechain of Serine is protected by the protecting group PG1, wherein PG1 is a base stable side-chain protecting group
  MOD_RES (34)..(34): The C-terminal Glycine is amidated and is bound to Sieber resin through this amine group

SEQ ID NO: 18 - protein, 34 aa, Artificial Sequence; identical to SEQ ID NO: 1
  Synthetic Construct
  MOD_RES (1)..(1): N-terminal Histidine is protected by Boc
  MOD_RES (1)..(1): Sidechain of Histidine is protected by Dnp
  MISC_FEATURE (2)..(2): Xaa at position 2 is Aib
  MOD_RES (3)..(3): Sidechain of Glutamine is protected by Trityl
  MOD_RES (5)..(5): Sidechain of Threonine is protected by t-Butyl
  MOD_RES (7)..(7): Sidechain of Threonine is protected by t-Butyl
  MOD_RES (8)..(8): Sidechain of Serine is protected by t-Butyl
  MOD_RES (9)..(9): Sidechain of Aspartic acid is protected by t-Butyl
  MOD_RES (10)..(10): Sidechain of Tyrosine is protected by t-Butyl
  MOD_RES (11)..(11): Sidechain of Serine is protected by t-Butyl
  MOD_RES (12)..(12): Sidechain of Lysine is protected by Boc
  MOD_RES (13)..(13): Sidechain of Tyrosine is protected by t-Butyl
  MOD_RES (15)..(15): Sidechain of Aspartic acid is protected by t-Butyl
  MOD_RES (16)..(16): Sidechain of Glutamic acid is protected by t-Butyl
  MOD_RES (17)..(17): Sidechain of Lysine is protected by Boc
  MOD_RES (18)..(18): Sidechain of Lysine is protected by Boc
  MOD_RES (21)..(21): Sidechain of Glutamic acid is protected by t-Butyl
  MOD_RES (24)..(24): Sidechain of Glutamic acid is protected by t-Butyl
  MOD_RES (25)..(25): Sidechain of Tryptophan is protected by Boc
  MOD_RES (28)..(28): Sidechain of Glutamic acid is protected by t-Butyl
  MOD_RES (32)..(32): Sidechain of Serine is protected by t-Butyl
  MOD_RES (33)..(33): Sidechain of Serine is protected by t-Butyl
  MOD_RES (34)..(34): The C-terminal Glycine is amidated and is bound to Sieber resin through this amine group

SEQ ID NO: 19 - protein, 34 aa, Artificial Sequence; identical to SEQ ID NO: 1
  Synthetic Construct
  MOD_RES (1)..(1): N-terminal Histidine is protected by Boc
  MOD_RES (1)..(1): Sidechain of Histidine is protected by Dnp
  MISC_FEATURE (2)..(2): Xaa at position 2 is Aib
  MOD_RES (3)..(3): Sidechain of Glutamine is protected by Trityl
  MOD_RES (5)..(5): Sidechain of Threonine is protected by t-Butyl
  MOD_RES (7)..(7): Sidechain of Threonine is protected by t-Butyl
  MOD_RES (8)..(8): Sidechain of Serine is protected by t-Butyl
  MOD_RES (9)..(9): Sidechain of Aspartic acid is protected by t-Butyl
  MOD_RES (10)..(10): Sidechain of Tyrosine is protected by t-Butyl
  MOD_RES (11)..(11): Sidechain of Serine is protected by t-Butyl
  MOD_RES (12)..(12): Sidechain of Lysine is protected by Boc
  MOD_RES (13)..(13): Sidechain of Tyrosine is protected by t-Butyl
  MOD_RES (15)..(15): Sidechain of Aspartic acid is protected by t-Butyl
  MOD_RES (16)..(16): Sidechain of Glutamic acid is protected by t-Butyl
  MOD_RES (17)..(17): Sidechain of Lysine is protected by Boc
  MOD_RES (18)..(18): Sidechain of Lysine is protected by Boc
  MOD_RES (20)..(20): Lysine at position 20 is chemically modified through conjugation to the epsilon-amino group of the K side-chain with (2-[2-(2-Amino-ethoxy)-ethoxy]-acetyl)2-(gamma- Glu(t-butyl))-CO-(CH2)18-CO2-(t-butyl)
  MOD_RES (21)..(21): Sidechain of Glutamic acid is protected by t-Butyl
  MOD_RES (24)..(24): Sidechain of Glutamic acid is protected by t-Butyl
  MOD_RES (25)..(25): Sidechain of Tryptophan is protected by Boc
  MOD_RES (28)..(28): Sidechain of Glutamic acid is protected by t-Butyl
  MOD_RES (32)..(32): Sidechain of Serine is protected by t-Butyl
  MOD_RES (33)..(33): Sidechain of Serine is protected by t-Butyl
  MOD_RES (34)..(34): The C-terminal Glycine is amidated and is bound to Sieber resin through this amine group

(19) United States
(12) Patent Application Publication
(10) Pub. No.: US 2023/0220000 A1
(43) Pub. Date: Jul. 13, 2023

(54) PROCESS FOR PREPARING A GLP-1/GLUCAGON DUAL AGONIST

(71) Applicant: Eli Lilly and Company, Indianapolis, IN (US)

(72) Inventors: Michael Edward KOBIERSKI, Greenwood, IN (US); Michael Eugene KOPACH, Greenwood, IN (US)

(21) Appl. No.: 18/000,853

(22) PCT Filed: Jun. 11, 2021
(86) PCT No.: PCT/US2021/036914

§ 371 Date: Dec. 6, 2022

Related U.S. Application Data
(60) Provisional application No. 63/038,363, filed on Jun. 12, 2020.

(51) Int. Cl. C07K 1/06; C07K 14/605; C07K 5/10; C07K 1/04

(52) U.S. Cl. CPC C07K 1/06; C07K 14/605; C07K 5/10; C07K 1/04

(57) ABSTRACT

The present invention provides processes and compounds for the preparation of glucagon and GLP-1 co-agonist compounds that are useful in the treatment of type 2 diabetes, obesity, nonalcoholic fatty liver disease (NAFLD) and/or nonalcoholic steatohepatitis (NASH).

The present invention provides processes for making a glucagon (Gcg) and GLP-1 dual agonist peptide, or a pharmaceutically acceptable salt thereof.

Over the past several decades, the prevalence of diabetes has continued to rise. Type 2 diabetes mellitus (T2D) is the most common form of diabetes accounting for approximately 90% of all diabetes. T2D is characterized by high blood glucose levels caused by insulin resistance. Uncontrolled diabetes leads to several conditions that impact morbidity and mortality of patients. The leading cause of death for diabetic patients is cardiovascular complications. One of the main risk factors for type 2 diabetes is obesity. The majority of T2D patients (˜90%) are overweight or obese. It is documented that a decrease in body adiposity will lead to improvement in obesity-associated co-morbidities including hyperglycaemia and cardiovascular events. Therefore, therapies effective in glucose control and weight reduction are needed for better disease management.

Gcg helps maintain the level of glucose in the blood by binding to Gcg receptors on hepatocytes, causing the liver to release glucose—stored in the form of glycogen—through glycogenolysis. As these stores become depleted, Gcg stimulates the liver to synthesize additional glucose by gluconeogenesis. This glucose is released into the bloodstream, preventing the development of hypoglycaemia.

GLP-1 has different biological activities compared to Gcg. The actions of GLP-1 include stimulation of insulin synthesis and secretion, inhibition of Gcg secretion and inhibition of food intake. GLP-1 has been shown to reduce hyperglycaemia in diabetics. Several GLP-1 agonists have been approved for use in the treatment of T2D in humans, including exenatide, liraglutide, lixisenatide, albiglutide and dulaglutide. Such GLP-1 agonists are effective in glycaemic control with favourable effects on weight without the risk of hypoglycaemia. However, the weight loss is modest due to dose-dependent gastrointestinal side-effects.

Gcg and GLP-1 dual agonist peptides that may be useful in the treatment of T2D and obesity are described and claimed in U.S. Pat. No. 9,938,335 B2. A process for the production of such Gcg and GLP-1 dual agonist peptides is described therein.

There remains a need, however, for improved processes for production of Gcg and GLP-1 dual agonist peptides, such processes having a combination of advantages including commercially desired purity. Similarly, there is a need for efficient and environmentally “green” processes, including stable compounds to provide Gcg and GLP-1 dual agonist peptides with fewer or simpler purification steps. The preparation of large-scale, pharmaceutically-elegant Gcg and GLP-1 dual agonist peptides presents a number of technical challenges that may affect the overall yield and purity. There is also a need for processes to avoid the use of harsh reaction conditions that are incompatible with peptide synthesis.

The present invention seeks to meet these needs by providing novel processes useful in the manufacture of a Gcg and GLP-1 dual agonist peptide (SEQ ID NO:1), or a pharmaceutically acceptable salt thereof. The improved manufacturing processes of the present invention provide compounds and process reactions embodying a combination of advances, including an efficient route having fewer steps, while at the same time maintaining high quality and purity. Importantly, the improved processes and compounds decrease resource intensity.

The improved processes described herein provide various compounds useful for production of a Gcg and GLP-1 dual agonist peptide.

In particular, there is provided a process for the preparation of a compound of the following formula:

H₂N-H-Aib-Q-G-T-F-T-S-D-Y-S-K-Y-L-D-E-K-K-A-K-E-F-V-E-W-L-L-E-G-G-P-S-S-G-NH₂

wherein lysine (Lys/K) at position 20 is chemically modified by conjugation of the epsilon-amino group of the lysine side chain with ([2-(2-aminoethoxy)-ethoxy]-acetyl)₂-(γ-Glu)-CO—(CH₂)₁₈CO₂H (SEQ ID NO: 1),

and wherein said process comprises the steps of:
(i) solid-phase synthesis of a compound of the following formula:

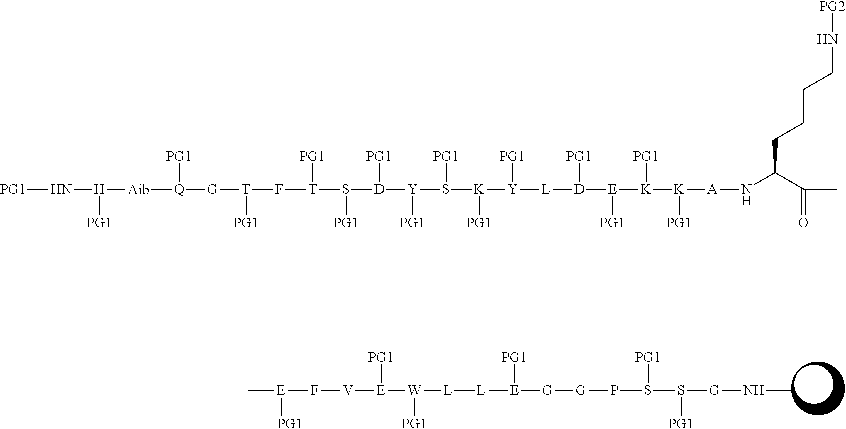

wherein PG1 is a base stable side-chain protecting group,
wherein the Thr at position 5 is optionally protected by PG1,
and wherein PG2 is an ivDde, Dde or Alloc side-chain protecting group (SEQ ID NO: 2);
(ii) selective acylation at Lys at position 20 (SEQ ID NO: 7) by selectively de-protecting said lysine and coupling the resulting Lys-NH₂ (SEQ ID NO: 5) with ^{t}BuO-C₂₀-γGlu(^{t}Bu)-AEEA-AEEA-OH;
(iii) cleavage of the compound from the solid support and removal of base stable side-chain protecting groups; and
(iv) purification of the compound (SEQ ID NO: 1).

Conventional preparation of a peptide compound wherein a side chain (e.g. fatty acid side chain) is built by individual couplings in a stepwise manner produce significant amounts of addition and deletion by-products. This results in an unfavourable purity profile that makes it challenging to purify the peptide compound of interest. Furthermore, low yields are typical when AEEA spacers are part of a side-chain built by conventional methods.

The selective deprotection of Lys at position 20 and subsequent acylation reaction proceeds with the de-protected 1-34 Lys-20-NH₂ peptide on resin backbone (SEQ ID NO: 4) coupled to the ^{t}BuO-C₂₀-γGlu(^{t}Bu)-AEEA-AEEA-OH sidechain as an intact fragment. This represents a novel on resin large fragment coupling. This approach provides an efficient and robust process for acylation of a peptide or protein wherein the compound is produced in high yield. Acylation occurs at lysine at position with >99% selectivity and minimal impurities. Selective deprotection and subsequent coupling results in a favorable impurity profile for the acylation reaction. Moreover, the improved acylation process facilitates an easier purification and isolation of the desired acylated peptide product that results in higher yields and purity.

Selective de-protection of the Lys at position 20 is facilitated by use of an ivDde, Dde or Alloc side-chain protecting group at position 20 and base stable side-chain protecting groups at other positions. De-protection conditions are selected wherein the ivDde, Dde or Alloc side-chain protecting group at position 20 is removed but the base-stable side-chain protecting groups (PG1) remain in place.

A variety of base-stable protecting groups are known in the art and may be used in the process of the present invention. In an embodiment of the present invention, the base-stable side-chain protecting groups PG1 used in the synthesis of the compound are (a) tert-butyloxycarbonyl (Boc) for Trp and Lys, (b) tert-butyl ester (O^{t}Bu) for Asp and Glu, (c) tert-butyl (^{t}Bu) for Ser, Thr and Tyr, (d) triphenylmethyl(trityl)(Trt) for Gln, and (e) Boc(Boc) or Boc(Dnp) for His.

In a preferred embodiment of the process of the present invention, the side-chain protecting group at Lys at position 20 is ivDde.

In an alternative embodiment of the process of the present invention, the side-chain protecting group at the Lys at position 20 is Dde.

Dde is a protecting group stable to most conventional bases and is, therefore, stable to Fmoc removal conditions. ivDde is a derivative of Dde and is also stable to Fmoc removal conditions. An additional advantage of ivDde is that its steric hindrance makes it less prone to migrate to other free Lys residues. Both Dde and ivDde are commonly removed by hydrazinolysis.

Preferably, when PG2 is ivDde or Dde, the Lys at position 20 is selectively de-protected by contacting the compound with a solution comprising hydrazine hydrate.

Further preferably, the solution comprises 1%-15% w/w hydrazine hydrate in DMF, NMP, NBP or DMSO.

Still further preferably, the solution comprises 8% w/w hydrazine hydrate in DMF.

In an alternative embodiment of the process of the present invention, the side-chain protecting group at the Lys at position 20 is Alloc.

Alloc is a base-labile protecting group. It is commonly removed by a palladium catalyst in the presence of a scavenger to capture the generated carbocation. The use of Alloc side-chain protecting group is compatible with the Boc/Bn and Fmoc/^{t}Bu strategies and allows tandem removal-acylation reactions when the palladium-catalyzed amino deblocking is performed in the presence of acylating agents. This approach prevents diketopiperazine (DKP) formation.

Preferably, when the side-chain protecting group at Lys at position 20 is Alloc, Lys at position 20 is selectively de-protected by contacting the compound with a palladium catalyst in the presence of scavengers,

Further preferably, the Alloc side-chain protecting group at Lys at position removed by contacting the compound with Pd(PPh₃)₄ in the presence of H₃N·BH3, Me₂NH·BH3, or PhSiH₃.

The de-protected (at position 20) compound may be washed, de-swelled, isolated, dried and packaged. The de-protected (at position 20) compound is re-swelled prior to coupling with sidechain

In a preferred embodiment of the process of the present invention, PG1 is Boc for Trp and Lys, O^{t}Bu for Asp and Glu, ^{t}Bu for Ser, Thr and Tyr, Trt for Gln and Boc(Boc) for His, PG2 is ivDde, and the solid-phase synthesis of the compound (SEQ ID NO: 3) of step (i) is performed on a Fmoc amide resin solid support and comprises Fmoc deprotection of the amide resin and sequential coupling of the following:
Fmoc-L-Gly-OH, Fmoc-L-Ser(^{t}Bu)-OH, Fmoc-L-Ser(^{t}Bu)-OH, Fmoc-L-Pro-OH, Fmoc-L-Gly-OH, Fmoc-L-Gly-OH, Fmoc-L-Glu(O^{t}Bu)-OH, Fmoc-L-Leu-OH, Fmoc-L-Leu-OH, Fmoc-L-Trp(Boc)-OH, Fmoc-L-Glu(O^{t}Bu)-OH, Fmoc-L-Val-OH, Fmoc-L-Phe-OH, Fmoc-L-Glu(O^{t}Bu)-OH, Fmoc-Lys(ivDde)-OH, Fmoc-L-Ala-OH, Fmoc-L-Lys(Boc)-OH, Fmoc-L-Lys(Boc)-OH, Fmoc-L-Glu(O^{t}Bu)-OH, Fmoc-L-Asp(O^{t}Bu)-OH, Fmoc-L-Leu-OH, Fmoc-L-Tyr(^{t}Bu)-OH, Fmoc-L-Lys(Boc)-OH, Fmoc-L-Ser(^{t}Bu)-OH, Fmoc-L-Tyr(^{t}Bu)-OH, Fmoc-L-Asp(O^{t}Bu)-OH, Fmoc-L-Ser(^{t}Bu)-OH, Fmoc-L-Thr(^{t}Bu)-OH, Fmoc-L-Phe-OH, Fmoc-Gly-Thr(ψ^{Me,Me}Pro)-OH, Fmoc-L-Gln(Trt)-OH, Fmoc-Aib-OH; and Boc-L-His(Boc)-OH.

In an alternative embodiment of the process of the present invention, PG1 is Boc(Dnp) for His and the solid-phase synthesis of the compound of step (i) is performed as described above.

Solid phase synthesis of the compound is performed on a Fmoc amide resin solid support wherein the first step is Fmoc deprotection of the amide resin followed by sequential coupling of the Fmoc amino acids of the peptide. A glycine-threonine pseudoproline dipeptide is used in place of individual Fmoc-L-Gly and Fmoc-L-Thr amino acids for coupling at positions 4 and 5. In these embodiments, the Thr residue at position 5 is reversibly protected as a proline-like acid-labile oxazolidine. As such, there is no requirement to protect that particular Thr residue with a PG1. A substantial benefit is realized in that the reaction proceeds to completion for the glycine-threonine pseudoproline dipeptide. In contrast, coupling individual Fmoc-L-Gly and Fmoc-L-Thr amino acids result in high levels of peptide impurities having a Thr5 deletion.

In an alternative preferred embodiment of the process of the present invention, PG1 is Boc for Trp and Lys, O^{t}Bu for Asp and Glu, Bu for Ser, Thr and Tyr, Trt for Gln, and Boc(Dnp) for His, PG2 is ivDde, and the solid-phase synthesis of the compound (SEQ ID NO: 4) of step (i) is performed on a Fmoc amide resin solid support and comprises Fmoc deprotection of the amide resin and sequential coupling of the following:
Fmoc-L-Gly-OH, Fmoc-L-Ser(^{t}Bu)-OH, Fmoc-L-Ser(^{t}Bu)-OH, Fmoc-L-Pro-OH, Fmoc-L-Gly-OH, Fmoc-L-Gly-OH, Fmoc-L-Glu(O^{t}Bu)-OH, Fmoc-L-Leu-OH, Fmoc-L-Leu-OH, Fmoc-L-Trp(Boc)-OH, Fmoc-L-Glu(O^{t}Bu)-OH, Fmoc-L-Val-OH, Fmoc-L-Phe-OH, Fmoc-L-Glu(O^{t}Bu)-OH, Fmoc-Lys(ivDde)-OH, Fmoc-L-Ala-OH, Fmoc-L-Lys(Boc)-OH, Fmoc-L-Lys(Boc)-OH, Fmoc-L-Glu(O^{t}Bu)-OH, Fmoc-L-Asp(O^{t}Bu)-OH, Fmoc-L-Leu-OH, Fmoc-L-Tyr(^{t}Bu)-OH, Fmoc-L-Lys(Boc)-OH, Fmoc-L-Ser(^{t}Bu)-OH, Fmoc-L-Tyr(^{t}Bu)-OH, Fmoc-L-Asp(O^{t}Bu)-OH, Fmoc-L-Ser(^{t}Bu)-OH, Fmoc-L-Thr(^{t}Bu)-OH, Fmoc-L-Phe-OH, and Boc-His(Dnp)-Aib-Gln(Trt)-Gly-Thr(^{t}Bu)-OH.

Solid phase synthesis of the compound is performed on a Fmoc amide resin solid support wherein the first step is Fmoc deprotection of the amide resin followed by sequential coupling of the Fmoc amino acids of the peptide. A Boc-His(Dnp)-Aib-Gln(Trt)-Gly-Thr(^{t}Bu)-OH pentamer (SEQ ID NO: 14) is coupled as a single fragment to Phe6 of the H₂N-6-34 intermediate (SEQ ID NO: 10). A substantial benefit realized by this preferred embodiment is improved purity due to minimization of histidine racemization.

The compound of SEQ ID NO: 4 may be selectively de-protected at the lysine at position 20 as described herein. The resulting compound has the following formula (SEQ ID NO: 18):

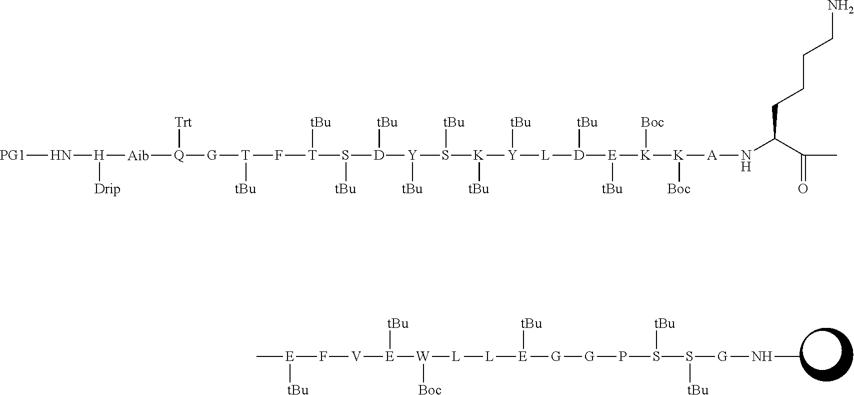

The compound of SEQ ID NO: 18 may be coupled with the ^{t}BuO-C₂₀-γGlu(^{t}Bu)-AEEA-AEEA-OH sidechain as an intact fragment as described herein. The resulting compound has the following formula (SEQ ID NO: 19):

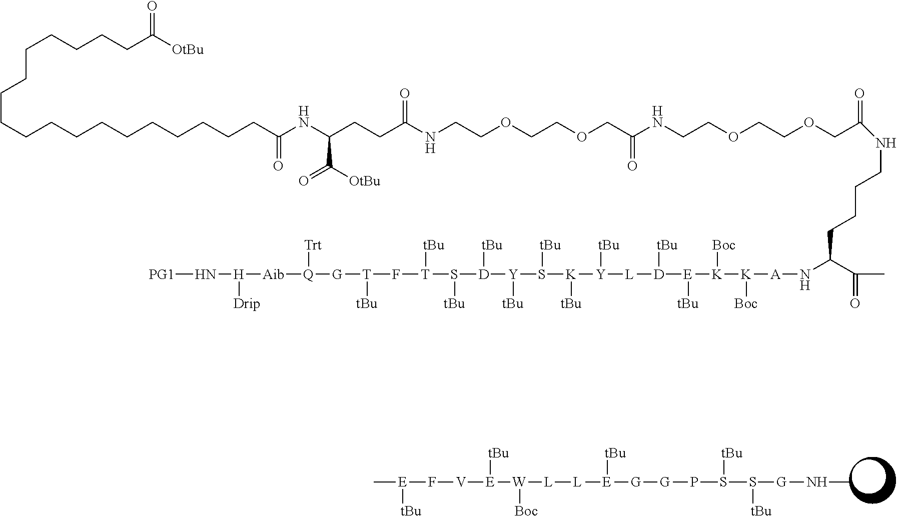

In a further alternative preferred embodiment of the process of the present invention. PG1 is: (a) Boc for Trp and Lys. (b) O^{t}Bu for Asp and Glu, (c) ^{t}Bu for Ser, Thr and Tyr, (d) Trt for Gln, and (e) Boc(Dnp) for His, PG2 is ivDde, and the solid-phase synthesis of the compound (SEQ ID NO: 4) of step (i) is performed on a Fmoc amide resin solid support and comprises Fmoc deprotection of the amide resin and sequential coupling of the following:
Fmoc-L-Gly-OH, Fmoc-L-Ser(^{t}Bu)-OH, Fmoc-L-Ser(Bu)-OH, Fmoc-L-Pro-OH, Fmoc-L-Gly-OH, Fmoc-L-Gly-OH, Fmoc-L-Glu(O^{t}Bu)-OH, Fmoc-L-Leu-OH, Fmoc-L-Leu-OH, Fmoc-L-Trp(Boc)-OH, Fmoc-L-Glu(O^{t}Bu)-OH, Fmoc-L-Val-OH, Fmoc-L-Phe-OH, Fmoc-L-Glu(O^{t}Bu)-OH, Fmoc-Lys(ivDde)-OH, Fmoc-L-Ala-OH, Fmoc-L-Lys(Boc)-OH, Fmoc-L-Lys(Boc)-OH, Fmoc-L-Glu(O^{t}Bu)-OH, Fmoc-L-Asp(O^{t}Bu)-OH, Fmoc-L-Leu-OH, Fmoc-L-Tyr(^{t}Bu)-OH, Fmoc-L-Lys(Boc)-OH, Fmoc-L-Ser(Bu)-OH, Fmoc-L-Tyr(^{t}Bu)-OH, Fmoc-L-Asp(O^{t}Bu)-OH, Fmoc-L-Ser(^{t}Bu)-OH, Fmoc-L-Thr(^{t}Bu)-OH, Fmoc-L-Phe-OH, Fmoc-L-Thr(^{t}Bu)-OH; and Boc-His(Dnp)-Aib-Gln(Trt)-Gly-OH.

Solid phase synthesis of the compound is performed on a Fmoc amide resin solid support wherein the first step is Fmoc deprotection of the amide resin followed by sequential coupling of the Fmoc amino acids of the peptide. A Boc-His(Dnp)-Aib-Gln(Trt)-Gly-OH tetramer (SEQ ID NO: 16) is coupled as a single fragment to Thr5 of the ₂HN-5-34 intermediate (SEQ ID NO: 12). A substantial benefit realized by this preferred embodiment is improved purity due to minimization of histidine racemization.

The compound of SEQ ID NO: 4 may be selectively de-protected at the lysine at position 20 as described herein. The resulting compound has the formula of SEQ ID NO: 18.

The compound of SEQ ID NO: 18 may be coupled with the ^{t}BuO-C₂₀-γGlu(^{t}Bu)-AEEA-AEEA-OH sidechain as an intact fragment as described herein. The resulting compound has the formula of SEQ ID NO: 19.

In a preferred embodiment of the process of the present invention, the resin solid support is a Fmoc amide resin solid support and the solid phase synthesis comprises Fmoc deprotection of the resin.

Further preferably, the Fmoc amide resin solid support is a Sieber resin.

In an embodiment of the present invention, step (iii) further comprises adjusting the pH of a solution comprising the cleaved and deprotected compound to 7.0-8.0, stirring for 1-24 hours, subsequently adjusting the pH of the solution to 1.0-3.0, and stirring for 1-24 hours.

Adjusting the pH to 7.0-8.0 neutralizes the solution and converts any depsi-peptide ester serine and threonine impurities to the desired compound.

Subsequent adjustment of the pH to 1.0-3.0 decarboxylates the Trp residue and converts the Trp CO₂ salt to the desired product.

In an embodiment of the process of the invention, the purification of the compound comprises subjecting the crude solution of the compound of step (iii) to chromatographic purification.

Preferably, the chromatographic purification is HPLC or reverse phase HPLC.

Still further preferably, the purification further comprises the steps of (i) adding the chromatographic eluent to a solution comprising aqueous sodium hydroxide or aqueous sodium bicarbonate to form a sodium salt of the compound in solution, (ii) precipitating the sodium salt of the compound from solution and (iii) filtering, washing and drying the precipitated sodium salt of the compound.

The sodium salt imparts improved solubility of the compound relative to the zwitterion or acetate forms. Furthermore, precipitation of the sodium salt of the compound replaces expensive lyophilization procedures.

In a further aspect of the present invention, there is provided a process for the preparation of a compound of the following formula:

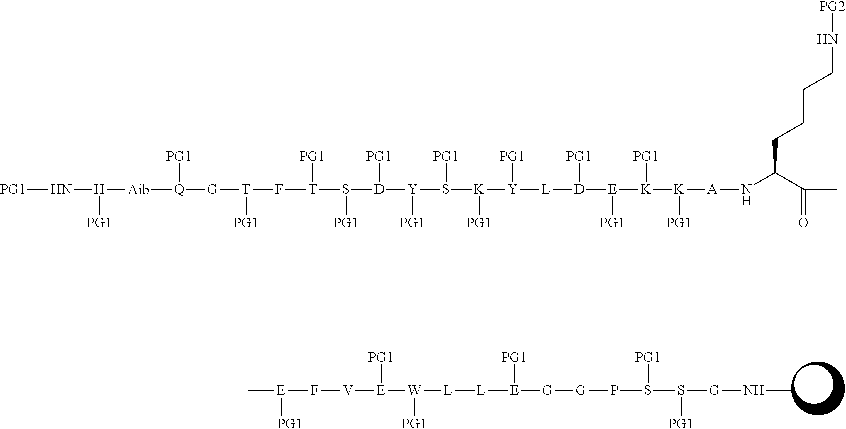

wherein PG1 is a base stable side-chain protecting group,

wherein PG2 is an ivDde, Dde or Alloc side-chain protecting group (SEQ ID NO: 17),

and wherein said process comprises the steps of:
(i) solid-phase synthesis of a compound of the following formula:

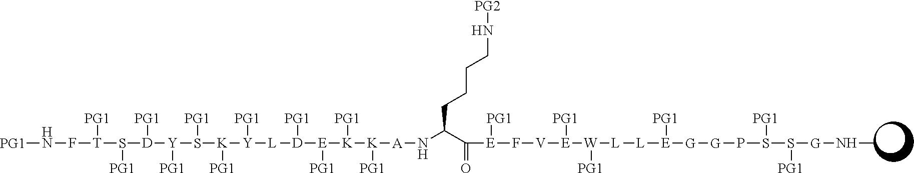

wherein PG1 is a base stable side-chain protecting group,
and wherein PG2 is an ivDde, Dde or Alloc side-chain protecting group (SEQ ID NO: 9); and
(ii) coupling the compound of step (i) with a pentamer of the following formula:

PG1-His(PG1)-Aib-Gln(PG1)-Gly-Thr(PG1)-OH
wherein PG1 is a base stable side-chain protecting group (SEQ ID NO: 13).

In a preferred embodiment of the process of the present invention, PG1 is Boc for Trp and Lys, O^{t}Bu for Asp and Glu, ^{t}Bu for Ser, Thr and Tyr, Trt for Gln, and Boc(Dnp) for His.

In a further preferred embodiment of the process of the present invention, PG2 is ivDde.

In an alternative preferred embodiment of the process of the process invention, PG2 is Dde.

In a further aspect of the present invention, there is provided a process for the preparation of a compound of the following formula:

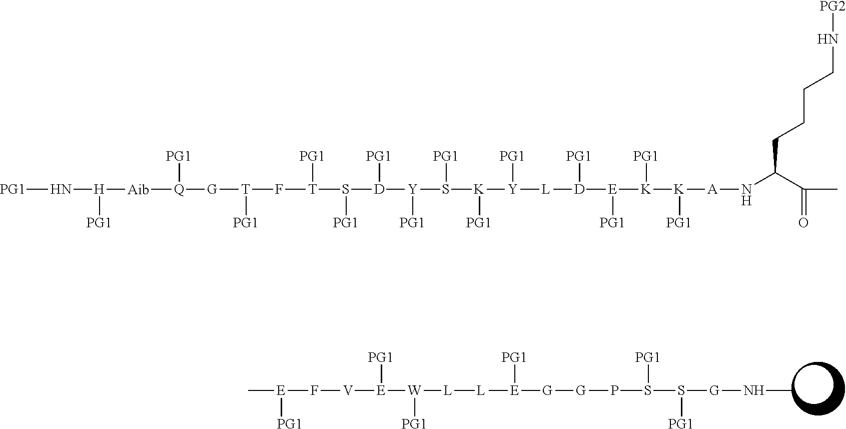

wherein PG1 is a base stable side-chain protecting group,
and wherein PG2 is an ivDde, Dde or Alloc side-chain protecting group (SEQ ID NO: 17)

said process comprising the steps of:
(i) solid-phase synthesis of a compound of the following formula:

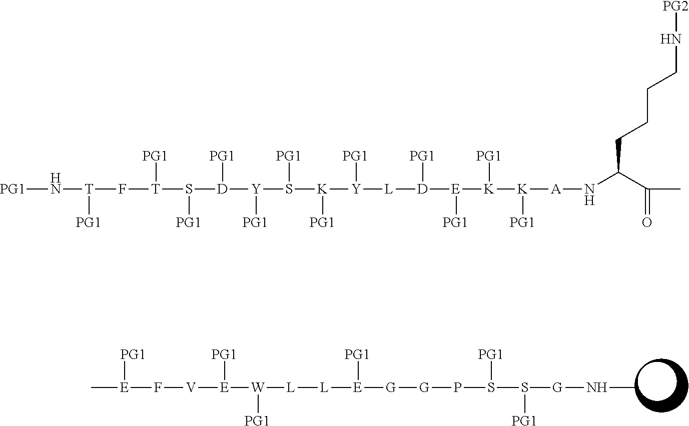

wherein PG1 is a base stable side-chain protecting group,
and wherein PG2 is an ivDde, Dde or Alloc side-chain protecting group (SEQ ID NO: 11); and
(ii) coupling the compound of step (i) with a tetramer of the following formula:

PG1-His(PG1)-Aib-Gln(PG1)-Gly-OH
wherein PG1 is a base stable side-chain protecting group (SEQ ID NO: 15).

In a preferred embodiment of the process of the present invention, PG1 is Boc for Trp and Lys, O^{t}Bu for Asp and Glu, ^{t}Bu for Ser, Thr and Tyr, Trt for Gln, and Boc(Dnp) for His.

In a further preferred embodiment of the process of the present invention, PG2 is ivDde.

In an alternative preferred embodiment of the process of the present invention, PG2 is Dde.

In a further aspect of the present invention, there is provided a process for the preparation of a sodium salt of the compound of the following formula:

H₂N-H-Aib-Q-G-T-F-T-S-D-Y-S-K-Y-L-D-E-K-K-A-K-E-F-V-E-W-L-L-E-G-G-P-S-S-G-NH₂

wherein lysine (Lys/K) at position 20 is chemically modified by conjugation of the epsilon-amino group of the lysine side chain with ([2-(2-aminoethoxy)-ethoxy]-acetyl)₂-(γ-Glu)-CO—(CH₂)₁₈CO₂H (SEQ ID NO: 1)

said process comprising the steps of:
(i) adding aqueous sodium hydroxide or aqueous sodium bicarbonate to a solution comprising the compound to form a sodium salt of the compound in solution;
(ii) precipitating the sodium salt of the compound from solution; and
(iii) filtering, washing and drying the precipitated sodium salt of the compound.

In a further aspect of the present invention, there is provided a compound having the following formula (SEQ ID NO: 3):

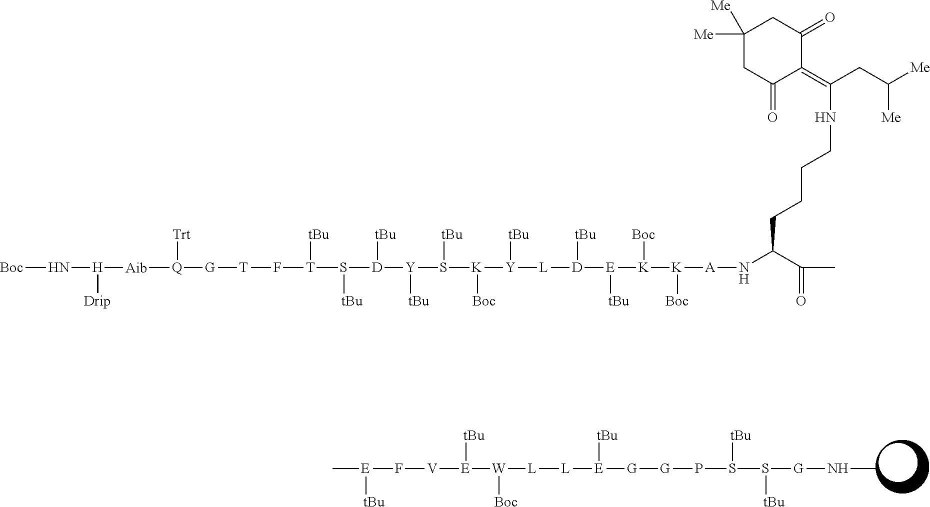

In a further aspect of the present invention, there is provided a compound having the following formula (SEQ ID NO: 4):

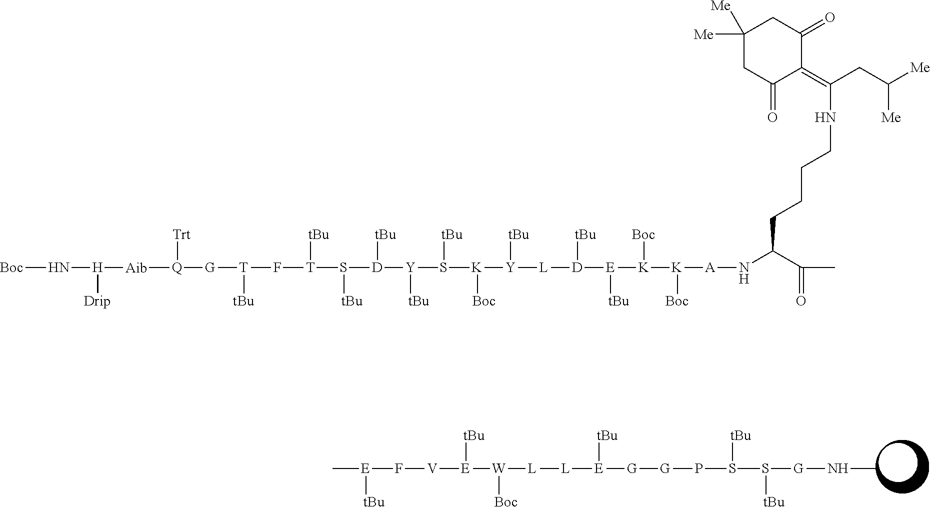

In a further aspect of the present invention, there is provided a compound having the following formula (SEQ ID NO: 10):

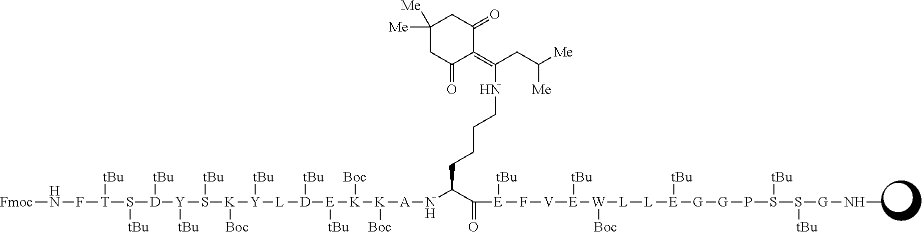

In a further aspect of the present invention there is provided a compound having the following formula (SEQ ID NO: 12):

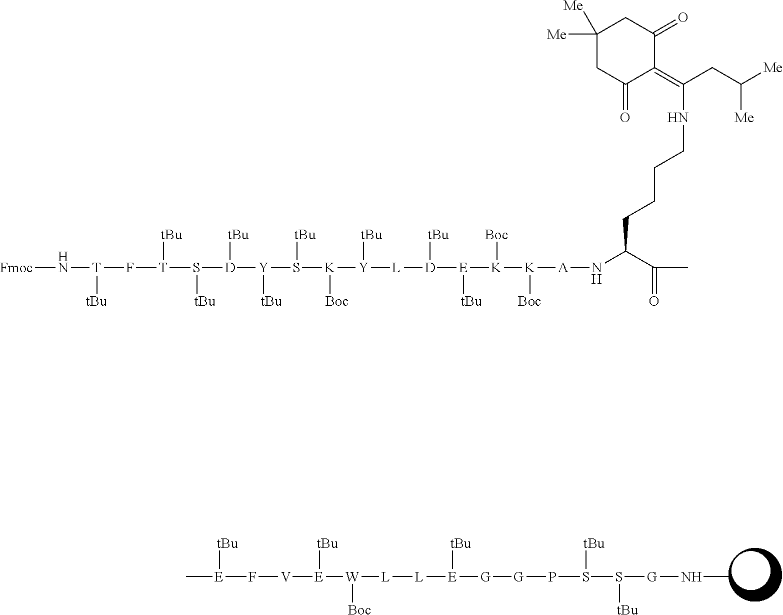

In a further aspect of the present invention, there is provided a compound having the following formula (SEQ ID NO: 13):

PG1-His(PG1)-Aib-Gln(PG1)-Gly-Thr(PG1)-OH

wherein PG1 is a base stable side-chain protecting group.

Preferably, PG1 is ^{t}Bu for Thr, Trt for Gln, and Boc(Dnp) for His.

In a further aspect of the present invention, there is provided a compound having the following formula (SEQ ID NO: 15):

PG1-His(PG1)-Aib-Gln(PG1)-Gly-OH

wherein PG1 is a base stable side-chain protecting group.

Preferably, PG1 is Trt for Gln and Boc(Dnp) for His.

### DETAILED DESCRIPTION

As used herein, the following abbreviations have the meanings as set forth herein: “SPPS” means Solid Phase Peptide Synthesis, “Fmoc” means fluorenylmethyloxycarbonyl chloride, “Boc” means tert-butyloxycarbonyl, “O^{t}Bu” means tert-butyl ester, “Bu” means tert-butyl, “Trt” means triphenylmethyl or trityl, “Dnp” means 2,4-dinitrophenyl, “ivDde” means 1-(4,4-Dimethyl-2,6-dioxocyclohex-1-ylidene)-3-methylbutyl, “Dde” means (1-(4,4-Dimethyl-2,6-dioxocyclohex-1-ylidene)-3-ethyl), “Alloc” means allyloxycarbonyl, “Pip” means piperidine, “DIC” means diisopropylcarbodiimide, “Oxyma” means Ethyl cyanohydroxyiminoacetate, “DCM” means dichloromethane, “IPA” means isopropanol, “MTBE” means methyl-tert-butyl ether, “TFA” means trifluoroacetic acid, “TIPS” means triisopropylsilane, “DTT” means dithiothreitol, “UPLC” means Ultra High Performance Liquid Chromatography, “HATU” means (1-[bis(dimethylamino)methylene]-1H-1,2,3-triazolo[4,5-b]pyridinium 3-oxide hexafluorophosphate, “HFIP” means hexafluoroisopropanol, “CTC” means chlorotrityl, “AEEA” means 17-amino-10-oxo-3,6,12,15 tetraoxa-9-aza heptadecanoic acid “TMSA” means trimethylsilyalmide, “HOBt” means hydroxybenzotriazole, and “API” means active pharmaceutical ingredient, “PyBOP” means (benzotriazol-1-yloxy)tripyrrolidinophosphonium hexafluorophosphate), “^{t}BuO-C₂₀-γGlu(^{t}Bu)-AEEA-AEEA-OH” means (3,6,12,15-Tetraoxa-9,18-diazatricosanedioic acid, 22-[[20-(1,1-dimethylethoxy)-1,20-dioxoeicosyl]amino]-10,19-dioxo-, 2,3-(1,1-dimethylethyl) ester, (22S)), and “AEEA” means (8-amino-3,6-dioxaoctanoic acid).

The amino acid sequences of the present invention contain the standard single letter or three letter codes for the twenty naturally occurring amino acids. Additionally, “Aib” is alpha amino isobutyric acid.

The present invention is generally directed to a process for the preparation of a Gcg and GLP-1 dual agonist compound wherein the compound is synthesized by SPPS. SPPS incorporates several basic steps that are repeated as additional amino acids are added to a growing peptide chain. The “solid phase” refers to resin particles to which initial amino acids—and then the growing peptide chains—are at attached. Because the chains are attached to particles, the chains can be handled as if they were a collection of solid particles (particularly for washing and separation—e.g., filtration-steps), and thus making the overall process easier in many cases than pure solution synthesis.

There are several suitable resins for building the peptide compounds presented herein. For example, Sieber and Rink amide resins are well known for preparing peptides. Alternative resins, however, may be selected for the preparation of peptides described herein. For example, but not limited to, 2-CTC and related resins may be used to prepare a target peptide, followed by a C terminus amidation step.

The repeated steps of SPPS include deprotection, activation and coupling:
(i) Deprotection: before each cycle starts, the last acid on the peptide chain remains “protected”. As used herein, the term “protected” means that a protecting group is attached to at the indicated position, i.e., its “amino” end is connected to a functional group that protects the acid from unwanted reactions. A variety of protecting groups are well known, and alternative protecting groups may be suitable for a particular process. The “protecting group” is removed (the “deprotection” step) when the next amino acid is about to be added;
(ii) Activation: a compound (“activator”) is added to the reaction to produce an intermediate amino acid species that is more likely to couple to the deprotected acid on the peptide chain.
(iii) Coupling: the activated species connects to the existing peptide chain.

One of the most commonly used and studied activation methods for peptide synthesis is based on the use of carbodiimides. A carbodiimide contains two slightly basic nitrogen atoms which will react with the carboxylic acid of an amino acid derivative to form a highly reactive O-acylisourea compound. The formed O-acylisourea can then immediately react with an amine to form a peptide bond. Alternatively, the O-acylisourea can be converted into other reactive species. Some of these alternative reactions of O-acylisourea, however, promote undesirable pathways that may or may not lead to peptide bond formation. Conversion to the unreactive N-acylurea prevents coupling, while epimerization of an activated chiral amino acid can occur through oxazolone formation. A more desirable highly reactive symmetrical anhydride can be formed by using excess amino acid compared to the carbodiimide. This approach, however, undesirably consumes an additional amino acid equivalent.

A significant improvement for carbodiimide activation methods occurred with the incorporation of 1-hydroxybenzotriazole (HOBt) as an additive during carbodiimide activation. HOBt quickly converts the O-acylisourea into an OBt ester that is highly reactive, but avoids undesirable N-acylisourea and oxazolone formation. HOBt is a hazardous reagent that is undesirable for use in large scale commercial manufacturing. Other additives can be used in place of HOBt such as ethyl 2-cyano-2-(hydroxyimino)acetate (Oxyma, OxymaPure, ECHA) or 1-hydroxy-2,5-pyrrolidinedione (NHS).

In respect of the processes of the present invention, the preferred activation system is DIC/Oxyma in DMF. Preferably, the ratio of amino acid:Oxyma:DIC is 2.0:2.0:2.2. All charges are based on the limiting reagent which is the amide resin. The Oxyma based system improves purity and eliminates downstream aggregation and impurity issues observed in the purification step, in particular chromatographic purification. Suitable solvents include DMF, NMP and NBP. DMF is the preferred solvent system as it is significantly cheaper.

More generally in respect of the processes of the present invention, the SPPS builds are preferably accomplished using standard Fmoc peptide chemistry techniques employing sequential couplings with an automated peptide synthesizer. The preferred resin is a Sieber amide resin. DMF is the preferred solvent system and the resin is swelled with DMF. De-protected of the resin is preferably achieved using 20% piperidine (Pip)/DMF (3×30 min). Subsequent Fmoc de-protections preferably use 20% Pip/DMF (9 ml/g resin) 3×30 min treatments. 4×30 min treatments are preferably used for more difficult couplings. After deprotection, the resin is washed with preferably 6×2 min, 10 volume DMF washes. Amino acid pre-activation preferably uses DIC/Oxyma/DMF solutions at room temp for 30 min. Coupling of the activated amino acid to the resin bound peptide occurs for a specified time for each individual amino acid. Solvent washing with preferably 6×2 min 10 volumes DMF is performed after each coupling.

For isolation of the final product, the resin bound product is preferably washed 5×2 min with 10 volume DCM to remove DMF. The resin is preferably washed with 2×2 min 10 volume IPA to remove DCM, washed 5×2 min 10 volume methyl-tert-butyl ether (MTBE), then the product is dried at 40° C. under vacuum. The resin bound product is stored cold (−20° C.).

For analysis, the peptide is cleaved from the resin with an acidic cocktail preferably consisting of TFA/H₂O/TIPS/DTT in the following ratio: (0.93 v/0.04 v/0.03 v/0.03 w). The resin is preferably swelled with DCM (4-5 mL, 3×30 min) and drained. The cleavage cocktail (4-5 mL) is added to the pre-swelled resin and the suspension is stirred for 2 hr at room temp. The solution is filtered then the resin is preferably washed with a small amount of DCM and combined with the cleavage solution. The resulting solution is preferably poured into 7-10 volumes of cold (0° C.) methyl-tert-butyl ether (MTBE). The suspension is preferably aged for 30 min at 0° C. then the resulting precipitate is centrifuged and the clear solution is decanted. The residue is preferably suspended in the same volume of MTBE, and the resulting suspension is again centrifuged and decanted. After decanting the clear MTBE solution of the precipitated peptide is dried in vacuo at 40° C. overnight.

The present invention is directed to novel compounds and processes useful for the synthesis of compounds disclosed herein, or a pharmaceutically acceptable salt thereof, in particular a sodium salt. The novel processes and compounds are illustrated in the Examples below. The reagents and starting materials are readily available to one of ordinary skill in the art. It is understood that these Examples are not intended to be limiting to the scope of the invention in any way.

### Example 1: Preparation of the Compound of SEQ ID NO: 1

### Synthesis of Preparation 1

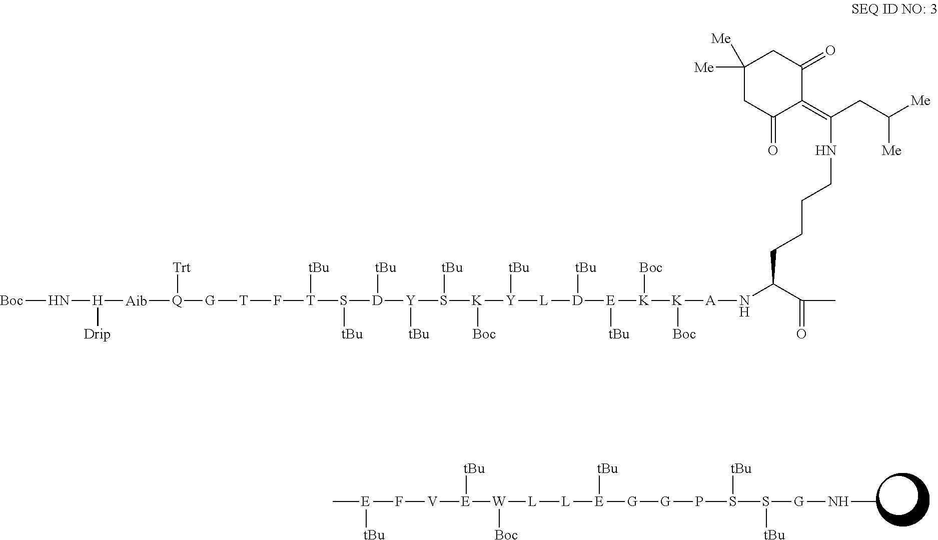

A Fmoc Sieber resin (0.6-0.8 mmol/g) is charged to a reactor, is swelled with DMF, stirred for 2 hours, then DMF filtered off from the resin. The resin is then washed with DMF twice. The Fmoc-protected resin is then de-protected using 20% Pip/DMF treatments at 9 ml/g resin. Sampling to verify Fmoc removal is performed after the last Pip/DMF treatment to confirm >99% Fmoc removal via UV analysis (IPC target <1% Fmoc remaining). After the final 20% w/w Pip/DMF treatment, the resin bed is washed multiple times with DMF (e.g. 6×2 min, 10 volume DMF washes at 9 ml/g resin). The peptide backbone is built out using the following conditions for each amino acid coupling and deprotection:

| Cycle | Amino acid | SPPS conditions |
|---|---|---|
| 1 | Fmoc-L-Gly-OH | (i) 3/4 × 30 min De-Fmoc cycles, |
| | | (ii) 6 × 2 min post-dep DMF washes (9 ml/g resin), |
| | | (iii) 2.0 AA/2.2 DIC/2.0 Oxyma, in DMF (7.25 ml/g resin), room |
| | | temperature (rt), |
| | | (iv) 6 × 2 min, 10 volumes DMF (9 ml/g resin) post-coupling |
| | | washes |
| 2 | Fmoc-L- | (i) 3/4 × 30 min De-Fmoc cycles, |
| | Ser(^{t}Bu)-OH | (ii) 6 × 2 min post-dep DMF washes (9 ml/g resin), |
| | | (iii) 2.0 AA/2.2 DIC/2.0 Oxyma, in DMF (7.25 ml/g resin), rt., |
| | | (iv) 6 × 2 min, 10 volumes DMF (9 ml/g resin) post-coupling |
| | | washes |
| 3 | Fmoc-L- | (i) 3/4 × 30 min De-Fmoc cycles, |
| | Ser(^{t}Bu)-OH | (ii) 6 × 2 min post-dep DMF washes (9 ml/g resin), |
| | | (iii) 2.0 AA/2.2 DIC/2.0 Oxyma, in DMF (7.25 ml/g resin), rt., |
| | | (iv) 6 × 2 min, 10 volumes DMF (9 ml/g resin) post-coupling |
| | | washes |
| 4 | Fmoc-L-Pro-OH | (i) 3/4 × 30 min De-Fmoc cycles, |
| | | (ii) 6 × 2 min post-dep DMF washes (9 ml/g resin), |
| | | (iii) 2.0 AA/2.2 DIC/2.0 Oxyma, in DMF (7.25 ml/g resin), rt., |
| | | (iv) 6 × 2 min, 10 volumes DMF (9 ml/g resin) post-coupling |
| | | washes |
| 5 | Fmoc-L-Gly-OH | (i) 3/4 × 30 min De-Fmoc cycles, |
| | | (ii) 6 × 2 min post-dep DMF washes (9 ml/g resin), |
| | | (iii) 2.0 AA/2.2 DIC/2.0 Oxyma, in DMF (7.25 ml/g resin), rt., |
| | | (iv) 6 × 2 min, 10 volumes DMF (9 ml/g resin) post-coupling |
| | | washes |
| 6 | Fmoc-L-Gly-OH | (i) 3/4 × 30 min De-Fmoc cycles, |
| | | (ii) 6 × 2 min post-dep DMF washes (9 ml/g resin), |
| | | (iii) 2.0 AA/2.2 DIC/2.0 Oxyma, in DMF (7.25 ml/g resin), rt., |
| | | (iv) 6 × 2 min, 10 volumes DMF (9 ml/g resin) post-coupling |
| | | washes |
| 7 | Fmoc-L- | (i) 3/4 × 30 min De-Fmoc cycles, |
| | Glu(O^{t}Bu)-OH | (ii) 6 × 2 min post-dep DMF washes (9 ml/g resin), |
| | | (iii) 2.0 AA/2.2 DIC/2.0 Oxyma, in DMF (7.25 ml/g resin), rt., |
| | | (iv) 6 × 2 min, 10 volumes DMF (9 ml/g resin) post-coupling |
| | | washes |
| 8 | Fmoc-L-Leu-OH | (i) 3/4 × 30 min De-Fmoc cycles, |
| | | (ii) 6 × 2 min post-dep DMF washes (9 ml/g resin), |
| | | (iii) 2.0 AA/2.2 DIC/2.0 Oxyma, in DMF (7.25 ml/g resin), rt., |
| | | (iv) 6 × 2 min, 10 volumes DMF (9 ml/g resin) post-coupling |
| | | washes |
| 9 | Fmoc-L-Leu-OH | (i) 3/4 × 30 min De-Fmoc cycles, |
| | | (ii) 6 × 2 min post-dep DMF washes (9 ml/g resin), |
| | | (iii) 2.0 AA/2.2 DIC/2.0 Oxyma, in DMF (7.25 ml/g resin), rt., |
| | | (iv) 5 × 2 min, 10 volumes DMF (9 ml/g resin) post-coupling |
| | | washes |
| 10 | Fmoc-L- | (i) 3/4 × 30 min De-Fmoc cycles, |
| | Trp(Boc)-OH | (ii) 6 × 2 min post-dep DMF washes (9 ml/g resin), |
| | | (iii) 2.0 AA/2.2 DIC/2.0 Oxyma, in DMF (7.25 ml/g resin), rt., |
| | | (iv) 6 × 2 min, 10 volumes DMF (9 ml/g resin) post-coupling |
| | | washes |
| 11 | Fmoc-L- | (i) 3/4 × 30 min De-Fmoc cycles, |
| | Glu(O^{t}Bu)-OH | (ii) 6 × 2 min post-dep DMF washes (9 ml/g resin), |
| | | (iii) 2.0 AA/2.2 DIC/2.0 Oxyma, in DMF (7.25 ml/g resin), rt., |
| | | (iv) 6 × 2 min, 10 volumes DMF (9 ml/g resin) post-coupling |
| | | washes |
| 12 | Fmoc-L-Val-OH | (i) 3/4 × 30 min De-Fmoc cycles, |
| | | (ii) 6 × 2 min post-dep DMF washes (9 ml/g resin), |
| | | (iii) 2.0 AA/2.2 DIC/2.0 Oxyma, in DMF (7.25 ml/g resin), rt., |
| | | (iv) 6 × 2 min, 10 volumes DMF (9 ml/g resin) post-coupling |
| | | washes |
| 13 | Fmoc-L-Phe-OH | (i) 3/4 × 30 min De-Fmoc cycles, |
| | | (ii) 6 × 2 min post-dep DMF washes (9 ml/g resin), |
| | | (iii) 2.0 AA/2.2 DIC/2.0 Oxyma, in DMF (7.25 ml/g resin), rt., |
| | | (iv) 6 × 2 min, 10 volumes DMF (9 ml/g resin) post-coupling |
| | | washes |
| 14 | Fmoc-L- | (i) 3/4 × 30 min De-Fmoc cycles, |
| | Glu(O^{t}Bu)-OH | (ii) 6 × 2 min post-dep DMF washes (9 ml/g resin), |
| | | (iii) 2.0 AA/2.2 DIC/2.0 Oxyma, in DMF (7.25 ml/g resin), rt., |
| | | (iv) 6 × 2 min, 10 volumes DMF (9 ml/g resin) post-coupling |
| | | washes |
| 15 | Fmoc- | (i) 3/4 × 30 min De-Fmoc cycles, |
| | Lys(ivDde)-OH | (ii) 6 × 2 min post-dep DMF washes (9 ml/g resin), |
| | | (iii) 8% hydrazine/DMF (9 ml/g resin), |
| | | (iv) 6 × 2 min, 10 volumes DMF (9 ml/g resin) post-coupling |
| | | washes |
| 16 | Fmoc-L-Ala-OH | (i) 3/4 × 30 min De-Fmoc cycles, |
| | | (ii) 6 × 2 min post-dep DMF washes (9 ml/g resin), |
| | | (iii) 2.0 AA/2.2 DIC/2.0 Oxyma, in DMF (7.25 ml/g resin), rt., |
| | | (iv) 6 × 2 min, 10 volumes DMF (9 ml/g resin) post-coupling |
| | | washes |
| 17 | Fmoc-L- | (i) 3/4 × 30 min De-Fmoc cycles, |
| | Lys(Boc)-OH | (ii) 6 × 2 min post-dep DMF washes (9 ml/g resin), |
| | | (iii) 2.0 AA/2.2 DIC/2.0 Oxyma, in DMF (7.25 ml/g resin), rt., |
| | | (iv) 6 × 2 min, 10 volumes DMF (9 ml/g resin) post-coupling |
| | | washes |
| 18 | Fmoc-L- | (i) 3/4 × 30 min De-Fmoc cycles, |
| | Lys(Boc)-OH | (ii) 6 × 2 min post-dep DMF washes (9 ml/g resin), |
| | | (iii) 2.0 AA/2.2 DIC/2.0 Oxyma, in DMF (7.25 ml/g resin), rt., |
| | | (iv) 6 × 2 min, 10 volumes DMF (9 ml/g resin) post-coupling |
| | | washes |
| 19 | Fmoc-L- | (i) 3/4 × 30 min De-Fmoc cycles, |
| | Glu(O^{t}Bu)-OH | (ii) 6 × 2 min post-dep DMF washes (9 ml/g resin), |
| | | (iii) 2.0 AA/2.2 DIC/2.0 Oxyma, in DMF (7.25 ml/g resin), rt., |
| | | (iv) 6 × 2 min, 10 volumes DMF (9 ml/g resin) post-coupling |
| | | washes |
| 20 | Fmoc-L- | (i) 3/4 × 30 min De-Fmoc cycles, |
| | Asp(O^{t}Bu)-OH | (ii) 6 × 2 min post-dep DMF washes (9 ml/g resin), |
| | | (iii) 2.0 AA/2.2 DIC/2.0 Oxyma, in DMF (7.25 ml/g resin), rt., |
| | | (iv) 6 × 2 min, 10 volumes DMF (9 ml/g resin) post-coupling |
| | | washes |
| 21 | Fmoc-L-Leu-OH | (i) 3/4 × 30 min De-Fmoc cycles, |
| | | (ii) 6 × 2 min post-dep DMF washes (9 ml/g resin), |
| | | (iii) 2.0 AA/2.2 DIC/2.0 Oxyma, in DMF (7.25 ml/g resin), rt., |
| | | (iv) 6 × 2 min, 10 volumes DMF (9 ml/g resin) post-coupling |
| | | washes |
| 22 | Fmoc-L- | (i) 3/4 × 30 min De-Fmoc cycles, |
| | Tyr(^{t}Bu)-OH | (ii) 6 × 2 min post-dep DMF washes (9 ml/g resin), |
| | | (iii) 2.0 AA/2.2 DIC/2.0 Oxyma, in DMF (7.25 ml/g resin), rt., |
| | | (iv) 6 × 2 min, 10 volumes DMF (9 ml/g resin) post-coupling |
| | | washes |
| 23 | Fmoc-L- | (i) 3/4 × 30 min De-Fmoc cycles, |
| | Lys(Boc)-OH | (ii) 6 × 2 min post-dep DMF washes (9 ml/g resin), |
| | | (iii) 2.0 AA/2.2 DIC/2.0 Oxyma, in DMF (7.25 ml/g resin), rt., |
| | | (iv) 6 × 2 min, 10 volumes DMF (9 ml/g resin) post-coupling |
| | | washes |
| 24 | Fmoc-L- | (i) 3/4 × 30 min De-Fmoc cycles, |
| | Ser(^{t}Bu)-OH | (ii) 6 × 2 min post-dep DMF washes (9 ml/g resin), |
| | | (iii) 2.0 AA/2.2 DIC/2.0 Oxyma, in DMF (7.25 ml/g resin), rt., |
| | | (iv) 6 × 2 min, 10 volumes DMF (9 ml/g resin) post-coupling |
| | | washes |
| 25 | Fmoc-L- | (i) 3/4 × 30 min De-Fmoc cycles, |
| | Tyr(^{t}Bu)-OH | (ii) 6 × 2 min post-dep DMF washes (9 ml/g resin), |
| | | (iii) 2.0 AA/2.2 DIC/2.0 Oxyma, in DMF (7.25 ml/g resin), rt., |
| | | (iv) 6 × 2 min, 10 volumes DMF (9 ml/g resin) post-coupling |
| | | washes |
| 26 | Fmoc-L- | (i) 3/4 × 30 min De-Fmoc cycles, |
| | Asp(O^{t}Bu)-OH | (ii) 6 × 2 min post-dep DMF washes (9 ml/g resin), |
| | | (iii) 2.0 AA/2.2 DIC/2.0 Oxyma, in DMF (7.25 ml/g resin), rt., |
| | | (iv) 6 × 2 min, 10 volumes DMF (9 ml/g resin) post-coupling |
| | | washes |
| 27 | Fmoc-L- | (i) 3/4 × 30 min De-Fmoc cycles, |
| | Ser(^{t}Bu)-OH | (ii) 6 × 2 min post-dep DMF washes (9 ml/g resin), |
| | | (iii) 2.0 AA/2.2 DIC/2.0 Oxyma, in DMF (7.25 ml/g resin), rt., |
| | | (iv) 6 × 2 min, 10 volumes DMF (9 ml/g resin) post-coupling |
| | | washes |
| 28 | Fmoc-L- | (i) 3/4 × 30 min De-Fmoc cycles, |
| | Thr(^{t}Bu)-OH | (ii) 6 × 2 min post-dep DMF washes (9 ml/g resin), |
| | | (iii) 2.0 AA/2.2 DIC/2.0 Oxyma, in DMF (7.25 ml/g resin), rt., |
| | | (iv) 6 × 2 min, 10 volumes DMF (9 ml/g resin) post-coupling |
| | | washes |
| 29 | Fmoc-L-Phe-OH | (i) 3/4 × 30 min De-Fmoc cycles, |
| | | (ii) 6 × 2 min post-dep DMF washes (9 ml/g resin), |
| | | (iii) 2.0 AA/2.2 DIC/2.0 Oxyma, in DMF (7.25 ml/g resin), rt., |
| | | (iv) 6 × 2 min, 10 volumes DMF (9 ml/g resin) post-coupling |
| | | washes |
| 30 | Fmoc-Gly- | (i) 3/4 × 30 min De-Fmoc cycles, |
| | Thr(ψ^{Me,Me}Pro)- | (ii) 6 × 2 min post-dep DMF washes (9 ml/g resin), |
| | OH | (iii) 2.0 AA/2.2 DIC/2.0 Oxyma, in DMF (7.25 ml/g resin), rt., |
| | | (iv) 6 × 2 min, 10 volumes DMF (9 ml/g resin) post-coupling |
| | | washes |
| 31 | Fmoc-L- | (i) 3/4 × 30 min De-Fmoc cycles, |
| | Gln(Trt)-OH | (ii) 6 × 2 min post-dep DMF washes (9 ml/g resin), |
| | | (iii) 2.0 AA/2.2 DIC/2.0 Oxyma, in DMF (7.25 ml/g resin), rt., |
| | | (iv) 6 × 2 min, 10 volumes DMF (9 ml/g resin) post-coupling |
| | | washes |
| 32 | Fmoc-Aib-OH | (i) 3/4 × 30 min De-Fmoc cycles, |
| | | (ii) 6 × 2 min post-dep DMF washes (9 ml/g resin), |
| | | (iii) 2.0 AA/2.2 DIC/2.0 Oxyma, in DMF (7.25 ml/g resin), rt., |
| | | (iv) 6 × 2 min, 10 volumes DMF (9 ml/g resin) post-coupling |
| | | washes |
| 33 | Boc-L-His(Boc)- | (i) 3/4 × 30 min De-Fmoc cycles, |
| | OH | (ii) 6 × 2 min post-dep DMF washes (9 ml/g resin), |
| | | (iii) 2.0 AA/2.2 DIC/2.0 Oxyma, in DMF (7.25 ml/g resin), rt., |
| | | (iv) 6 × 2 min, 10 volumes DMF (9 ml/g resin) post-coupling |
| | | washes |

### Fmoc Deprotection:

Resin in the peptide reactor is treated with either three or four charges of the 20% v/v Pip/DMF solution. Each treatment is stirred on the resin for 30 min followed by filtration to complete Fmoc protecting group removal. After the final 20% v/v PIP/DMF treatment, the resin bed is washed a minimum of six times with DMF at the pre-specified DMF volume charge.

### Amino Acid Activation:

A pre-prepared solution of 12% w/w Oxyma Pure/DMF is charged to a reactor. The selected Fmoc amino acid is then added. The mixture is stirred at 20±5° C. until the Fmoc amino acid has completely dissolved. The Fmoc-AA/Oxyma Pure/DMF solutions are then cooled to 15±3° C. prior to activation to ensure the minor exothermic activation reaction is controlled and the resulting solution temperature is maintained in the range specified of 20±5° C. The amino acid solution is activated by DIC addition. The activated ester solution is stirred for 20-30 minutes prior to transfer of the solution to the reactor containing the peptide on resin compound.

### Coupling:

Upon completion of the activation step, the activated ester solution is transferred to the reactor containing deprotected peptide on resin to initiate the coupling reaction. The peptide coupling reaction is stirred at 20±5° C. for at least 4 hours. After the required stir time, the resin slurry is sampled for coupling completion (IPC). Sampling is repeated at specific intervals as needed until a passing IPC result is obtained. Re-coupling operations are performed, if necessary. When the coupling is complete, the peptide reactor solution contents are filtered then the peptide on resin compounds are washed several times with DMF to prepare for the next coupling.

A Gly-Thr pseudoproline dipeptide is used in place of individual Fmoc-L-Gly and Fmoc-L-Thr amino acids for coupling at positions 4 and 5. Fmoc-Gly-Thr[ψ(^{Me,Me})Pro]-OH is coupled to Phe (6) using the above-described coupling conditions.

### Alternative Synthesis of Preparation 1

An alternative synthesis of Preparation 1 utilizes HOBT in NMP as a substitute for Oxyma in DMF in the amino acid activation step. The activating agent is DIC. The ratio of amino acid to DIC to HOBT is 3.0:3.3:3.0 (3.0 AA/3.3 DIC/3.0 HOBT). The solvent system is NMP. NMP is the solvent system that is also used in the coupling and deprotection reactions in the alternative synthesis.

### Synthesis of Preparation 2

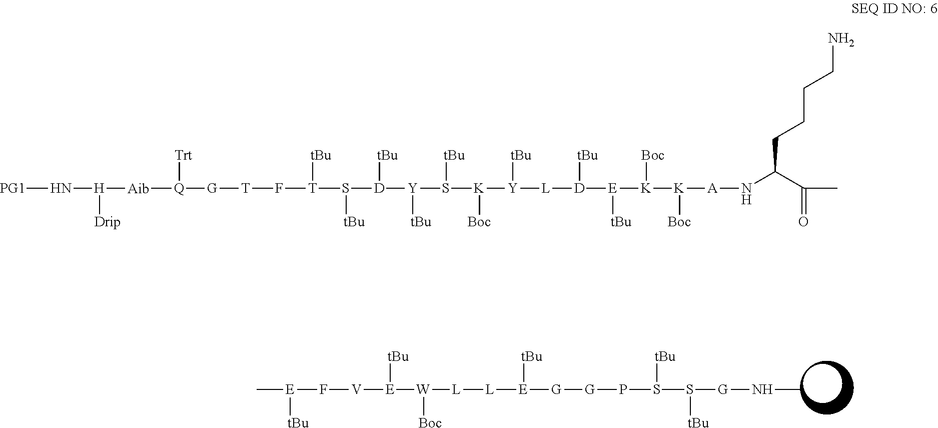

Lys (20) ivDde De-Protection:
A selective de-protection of the 1-34 Lys(20) ivDde group of the 34 amino acid full protected on resin Boc-His(1)-Gly(34) peptide backbone is performed. De-protection is achieved using 8% w/w hydrazine hydrate in DMF solution with stirring for 4 h at ambient temperature. The de-protection reaction is monitored by HPLC targeting an IPC limit of <1% of the 1-34 Lys(ivDde) component remaining after de-protection. The resulting peptide fragment (Preparation 2; SEQ ID NO: 3), is repetitively washed (8×) with DMF to completely remove residual hydrazine. The fully built Preparation 2 fragment is washed four times with IPA then dried at ≤40° C. until LOD of ≤1% is achieved. Preparation 2 is packaged and stored cold (−20° C.) prior to coupling with ^{t}BuO-C20-γGlu(^{t}Bu)-AEEA-AEEA-OH.

### Synthesis of Preparation 3

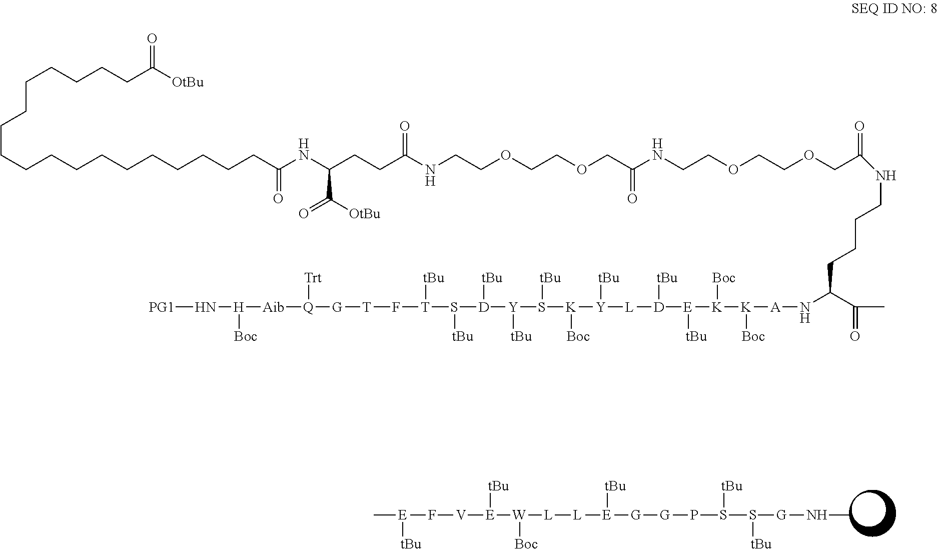

Coupling of ^{t}BuO-C₂₀-γGlu(Bu)-AEEA-AEEA-OH to Preparation 2:

Sidechain BuO-C₂₀-γGlu(^{t}Bu)-AEEA-AEEA-OH (2.0 equiv) and PyBOP (3.0 equiv) solids are charged to a reactor followed by 1:1 DMF/DCM and the mixture stirred until dissolution occurs. 2,4,6 Collidine (3.0 equiv.) is charged to initiate formation of the active ester species. The activated ester solution is stirred for 30 min prior to transfer to the reactor containing the Preparation 2 compound. The reaction slurry is stirred for 18 h at 35° C. The slurry is sampled for coupling completion (IPC) and sampling is repeated, if necessary, at specific intervals as needed to achieve passing IPC (≤1% Preparation 2) results.

When the coupling is complete, the solution contents are filtered to waste. The fully built Preparation 3 compound is washed multiple times with DMF, then IPA.

Preparation 3 is dried at ≤40° C. until LOD ≤1% is achieved. Preparation 3 is packaged and stored cold (−20° C.) prior to cleavage from resin.

By following the synthesis of Preparation 1, Preparation 2, and Preparation 3 as described above, 28 g Sieber resin (0.6 mmol/g) is processed into 85 g of on resin compound (i.e., Preparation 3)(73% yield).

### Alternative Synthesis of Preparation 3

The peptide backbone is built according to the alternative synthesis of Preparation 1 as described above. All Fmoc deprotections are performed using 20 wt % Pip/NMP. The post de-protection washes use DMF solvent. For coupling of N-terminus Boc-His-BOC-OH, a DEPT/DIEA activation system is used. The pre-formed activated esters are added to resin slurried in NMP.

After selective deprotection of Lys20 ivDde with hydrazine to form Preparation 2 as described above, four individual side chain couplings are sequentially performed to complete the resin bound build. Each cycle utilized the PyBOP/DIEA coupling reagent pair. Three of the side chain components are Fmoc-based reagents following the typical de-protection, coupling and DMF washing protocols. The final cycle uses the mono t-butyl protected twenty carbon fatty di-acid as the final segment coupled to the γGlu side chain. For this coupling, a 75:25 w/w toluene: NMP solvent mixture is used to ensure the fatty acid remained in solution throughout the coupling sequence.

By following the alternative synthesis of Preparation 1 and alternative synthesis of Preparation 3 as described above, 1.4 kg Sieber resin (0.6 mmol/g) is processed to 4.6 kg of on resin compound (i.e., Preparation 3)(79% yield).

### Synthesis of Preparation 4

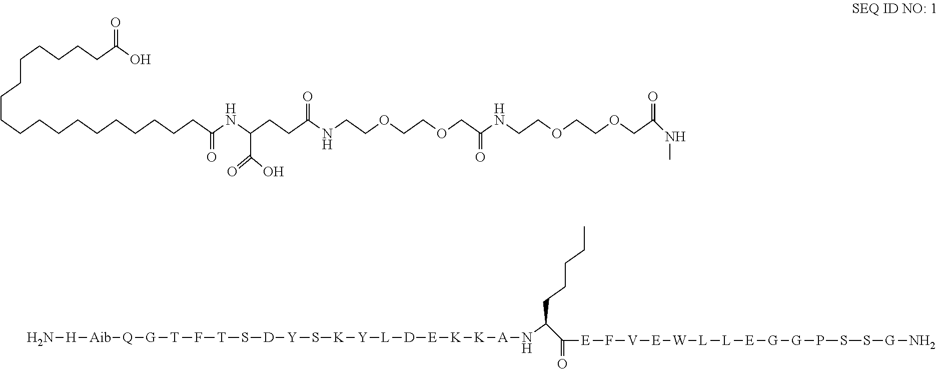

### Resin Cleavage/Deprotection:

A cleavage cocktail is prepared consisting of TFA, TIPS, DTT, DCM, and water. The cleavage cocktail is cooled to 15±5° C. Reagent charges are shown in the following table:

| | Solvent/ | Volume (per Resin | |
|---|---|---|---|
| Process step | Reagent | Bound charged) | |
| Cleavage cocktail | TFA | 7.16 | ml/g |
| | water | 0.34 | ml/g |
| | TIPS | 0.24 | ml/g |
| | DTT | 0.24 | g/g |
| | DCM | 0.75 | ml/g |
| Net cocktail charge | n/a | ~8.50 | ml/g |
| Spent resin wash | DCM | 3 | ml/g |
| Anti-solvent | MTBE | 14 | g/g |
| Vessel and cake washes | MTBE | 3 | g/g |

Preparation 3 is charged to a reactor followed by the cleavage cocktail. The mixture is stirred and maintained at 23° C. for 3 hour. The mixture is filtered then the spent resin is washed with DCM. The DCM wash filtrate is combined with the bulk de-protection solution and the contents cooled to ≤−10° C. MTBE is cooled to ≤−13° C. fed to the cold filtrate in two portions. The MTBE feed rate is controlled to maintain the crude solution internal temperature at ≤5° C. The initial MTBE charge constituted ˜ 45% of the total MTBE charge. A soft precipitate forms near the end of the MTBE addition but readily re-dissolved into solution. The precipitation solution is then re-cooled to an internal temperature of −15±5° C. The second MTBE addition is fed at a rate approximately 5-10 times the initial MTBE feed rate and constituted ˜55% of the total MTBE charge. The precipitation slurry internal temperature is maintained at ≤0° C. during the addition. The resulting slurry is aged at −8±3° C. for a minimum of 6 hours followed then warmed to 0±3° C. and aging for an additional 2 hours prior to isolation. The cold crude peptide slurry is filtered then the resulting wet-cake washed with MTBE. The Preparation 4 wet-cake is then dried to an IPC target LOD value of <1%.

By following the synthesis of Preparation 4 described above, Preparation 4 is produced with 44 wt % and 65% HPLC area percent purity. The contained yield based on Sieber resin is 47%.

### Purification

The zwitterionic form of Preparation 4 is purified by chromatography and subsequently lyophilized.

### Chromatography:

4.25 kg of Preparation 4 (41% potency, 1.71 kg active content) (prepared according to the alternative synthesis of Preparation 1 and alternative synthesis of Preparation 3 described above) is dissolved in 4/6/90 formic acid/acetonitrile/water solution to form a 10 mg/mL solution which is stirred for four hours to decarboxylate tryptophan prior to chromatography. The dissolved peptide is subsequently processed through reverse phase chromatography using 27 primary injections and 2 recycles on a 15 cm column to produce 671 kg total solution containing 1.43 kg of the compound of SEQ ID NO: 1 (93% purity and 83% yield). The compound of SEQ ID NO: 1 is further purified by additional reverse phase chromatography on a 15 cm column using 22 primary injections and 4 recycles to deliver 278 kg solution containing 1.19 kg of the compound of SEQ ID NO: 1 (98% purity, 93.6% yield). Concentration chromatography using Amberchrom resin is then performed with 4 primary injections to deliver 38.4 kg total solution with 1.16 kg active peptide content (98% purity, 93.6% yield).

### Lyophilization:

The chromatography concentration solution is heated to 35° C. then diluted with acetonitrile (50 volumes) at a feed rate of 100-150 g per minute. The dilute peptide solution is seeded with 5 g (95% purity) of the compound of SEQ ID NO: 1 (zwitterionic form) then stirred at 35° C. until precipitate forms. A second charge of acetonitrile (50 volumes) is added maintaining a temperature of 35° C. The resulting slurry is aged at 35° C. for 1 hour, cooled to 20° C. then aged a least one hour. The slurry is filtered, then the isolated product washed with acetonitrile and dried until <1% LOD achieved. The dry product is then humidified to remove any residual solvents. The humidified API powder is dissolved in 29 volumes of a 0.38% (w/w) solution of ammonium acetate in high purity water then 1.33 volumes of a 9.1% (w/w) solution of ammonium hydroxide in high purity water is added in aliquots to achieve dissolution and a final solution pH in the range of pH 8.2 to pH 8.6.

The aqueous solution of the compound is filtered through a 0.2 micron polyethersulfone filter while filling lyophilization trays to contain approximately 0.9 kg of aqueous solution per tray. The product is lyophilized according to an automated program which includes freezing the solutions at −40° C. Main lyophilization is performed at a temperature of −40° C. and vacuum of ˜100 mTorr. After primary lyophilization, a gradual ramp sequence is performed to elevate the shelf temperature from −40° C. to 0° C. Secondary drying is performed at approximately 15 mTorr and 20° C. to produce 412 g of the compound of SEQ ID NO: 1 as a white solid in 98% purity and 95% yield.

### Purification and Sodium Salt Synthesis

### Chromatography

First-pass HPLC purification is performed using 0.1/90/10, TFA/water/acetonitrile (v/v) mobile phase A, 0.1/10/90 (v/v) TFA/water/acetonitrile mobile phase B, and Kromasil 100-10-C8 stationary phase.

Second-pass HPLC purification is performed using 90/10 50 mM ammonium bicarbonate, pH 7.6/acetonitrile (v/v) mobile phase A (MP-A) and 10/90 50 mM ammonium bicarbonate, pH 7.6/acetonitrile (v/v) mobile phase B (MP-B) on Kromasil 100-10-C8 as stationary phase.

### Sodium Salt Synthesis

After the chromatography purification, the second pass composite solution is concentrated using 90% 50 mM ammonium acetate, pH 8.5/10% isopropyl alcohol (v/v) mobile phase A (MP-A), 10% 50 mM ammonium acetate, pH 8.5/90% isopropyl alcohol (v/v) mobile phase B (MP-B) and Amberchrom CG300-M stationary phase.

Aqueous sodium hydroxide solution is charged to the concentrate solution based on the molar equivalents of acid functionality present in the peptide molecule; an equal molar quantity of hydroxide (OH—) is added to neutralize the free carboxylic acid groups of the peptide. This is to be a maximum addition based on observed pH adjustment, which is targeted at pH≈9.0. The resulting peptide sodium salt is precipitated by the slow metered addition of acetonitrile (ACN) at 20° C. followed by aging and then seeding. Precipitation is completed by the subsequent gradual addition of additional ACN, at 20° C., to the diluted solution that is seeded with 1 wt % of the sodium salt of the compound of SEQ ID NO: 1. From the resultant precipitated slurry, the filtered solids are washed with additional ACN at ambient temperature to displace mother liquors. The precipitated solid is dried under vacuum to a final LOD (<1%) target limit. 10 g of the sodium salt of the compound of SEQ ID NO: 1 is produced in greater than 95.0% HPLC purity without any individual impurities higher than 1.0%. The overall process yield from Sieber resin is 25%.

### Example 2: Preparation of the Compound of SEQ ID NO: 10

### Synthesis of Preparation 5

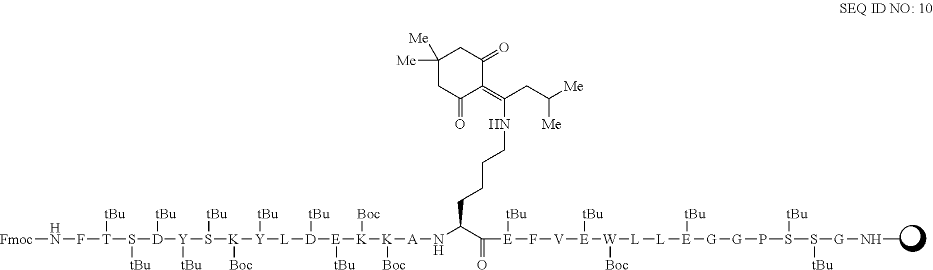

A Fmoc Sieber resin (0.6-0.8 mmol/g) is charged to a reactor is swelled with DMF, stirred for 2 hours, then DMF filtered off from the resin. The resin is then washed with DMF twice. The Fmoc-protected resin is then de-protected using 20% Pip/DMF treatments at 9 ml/g resin. Sampling to verify Fmoc removal is performed after the last Pip/DMF treatment to confirm >99% Fmoc removal via UV analysis (IPC target <1% Fmoc remaining). After the final 20% w/w Pip/DMF treatment, the resin bed is washed multiple times with DMF (e.g. 6×2 min, 10 volume DMF washes at 9 ml/g resin). The peptide backbone is built out using the following conditions for each amino acid coupling and deprotection:

| Cycle | Amino acid | SPPS conditions |
|---|---|---|
| 1 | Fmoc-L-Gly-OH | (i) 3/4 × 30 min De-Fmoc cycles, |
| | | (ii) 6 × 2 min post-dep DMF washes (9 ml/g resin), |
| | | (iii) 2.0 AA/2.2 DIC/2.0 Oxyma, in DMF (7.25 ml/g resin), room |
| | | temperature (rt). |
| | | (iv) 6 × 2 min, 10 volumes DMF (9 ml/g resin) post-coupling |
| | | washes |
| 2 | Fmoc-L- | (i) 3/4 × 30 min De-Fmoc cycles, |
| | Ser(^{t}Bu)-OH | (ii) 6 × 2 min post-dep DMF washes (9 ml/g resin), |
| | | (iii) 2.0 AA/2.2 DIC/2.0 Oxyma, in DMF (7.25 ml/g resin), rt., |
| | | (iv) 6 × 2 min, 10 volumes DMF (9 ml/g resin) post-coupling |
| | | washes |
| 3 | Fmoc-L- | (i) 3/4 × 30 min De-Fmoc cycles, |
| | Ser(^{t}Bu)-OH; | (ii) 6 × 2 min post-dep DMF washes (9 ml/g resin), |
| | | (iii) 2.0 AA/2.2 DIC/2.0 Oxyma, in DMF (7.25 ml/g resin), rt., |
| | | (iv) 6 × 2 min, 10 volumes DMF (9 ml/g resin) post-coupling |
| | | washes |
| 4 | Fmoc-L-Pro-OH | (i) 3/4 × 30 min De-Fmoc cycles, |
| | | (ii) 6 × 2 min post-dep DMF washes (9 ml/g resin), |
| | | (iii) 2.0 AA/2.2 DIC/2.0 Oxyma, in DMF (7.25 ml/g resin), rt., |
| | | (iv) 6 × 2 min, 10 volumes DMF (9 ml/g resin) post-coupling |
| | | washes |
| 5 | Fmoc-L-Gly-OH | (i) 3/4 × 30 min De-Fmoc cycles, |
| | | (ii) 6 × 2 min post-dep DMF washes (9 ml/g resin), |
| | | (iii) 2.0 AA/2.2 DIC/2.0 Oxyma, in DMF (7.25 ml/g resin), rt., |
| | | (iv) 6 × 2 min, 10 volumes DMF (9 ml/g resin) post-coupling |
| | | washes |
| 6 | Fmoc-L-Gly-OH | (i) 3/4 × 30 min De-Fmoc cycles, |
| | | (ii) 6 × 2 min post-dep DMF washes (9 ml/g resin), |
| | | (iii) 2.0 AA/2.2 DIC/2.0 Oxyma, in DMF (7.25 ml/g resin), rt., |
| | | (iv) 6 × 2 min, 10 volumes DMF (9 ml/g resin) post-coupling |
| | | washes |
| 7 | Fmoc-L- | (i) 3/4 × 30 min De-Fmoc cycles, |
| | Glu(O^{t}Bu)-OH | (ii) 6 × 2 min post-dep DMF washes (9 ml/g resin), |
| | | (iii) 2.0 AA/2.2 DIC/2.0 Oxyma, in DMF (7.25 ml/g resin), rt., |
| | | (iv) 6 × 2 min, 10 volumes DMF (9 ml/g resin) post-coupling |
| | | washes |
| 8 | Fmoc-L-Leu-OH | (i) 3/4 × 30 min De-Fmoc cycles, |
| | | (ii) 6 × 2 min post-dep DMF washes (9 ml/g resin), |
| | | (iii) 2.0 AA/2.2 DIC/2.0 Oxyma, in DMF (7.25 ml/g resin), rt., |
| | | (iv) 6 × 2 min, 10 volumes DMF (9 ml/g resin) post-coupling |
| | | washes |
| 9 | Fmoc-L-Leu-OH | (i) 3/4 × 30 min De-Fmoc cycles, |
| | | (ii) 6 × 2 min post-dep DMF washes (9 ml/g resin), |
| | | (iii) 2.0 AA/2.2 DIC/2.0 Oxyma, in DMF (7.25 ml/g resin), rt., |
| | | (iv) 5 × 2 min, 10 volumes DMF (9 ml/g resin) post-coupling |
| | | washes |
| 10 | Fmoc-L- | (i) 3/4 × 30 min De-Fmoc cycles, |
| | Trp(Boc)-OH | (ii) 6 × 2 min post-dep DMF washes (9 ml/g resin), |
| | | (iii) 2.0 AA/2.2 DIC/2.0 Oxyma, in DMF (7.25 ml/g resin), rt., |
| | | (iv) 6 × 2 min, 10 volumes DMF (9 ml/g resin) post-coupling |
| | | washes |
| 11 | Fmoc-L- | (i) 3/4 × 30 min De-Fmoc cycles, |
| | Glu(O^{t}Bu)-OH | (ii) 6 × 2 min post-dep DMF washes (9 ml/g resin), |
| | | (iii) 2.0 AA/2.2 DIC/2.0 Oxyma, in DMF (7.25 ml/g resin), rt., |
| | | (iv) 6 × 2 min, 10 volumes DMF (9 ml/g resin) post-coupling |
| | | washes |
| 12 | Fmoc-L-Val-OH | (i) 3/4 × 30 min De-Fmoc cycles, |
| | | (ii) 6 × 2 min post-dep DMF washes (9 ml/g resin), |
| | | (iii) 2.0 AA/2.2 DIC/2.0 Oxyma, in DMF (7.25 ml/g resin), rt., |
| | | (iv) 6 × 2 min, 10 volumes DMF (9 ml/g resin) post-coupling |
| | | washes |
| 13 | Fmoc-L-Phe-OH | (i) 3/4 × 30 min De-Fmoc cycles, |
| | | (ii) 6 × 2 min post-dep DMF washes (9 ml/g resin), |
| | | (iii) 2.0 AA/2.2 DIC/2.0 Oxyma, in DMF (7.25 ml/g resin), rt., |
| | | (iv) 6 × 2 min, 10 volumes DMF (9 ml/g resin) post-coupling |
| | | washes |
| 14 | Fmoc-L- | (i) 3/4 × 30 min De-Fmoc cycles, |
| | Glu(O^{t}Bu)-OH | (ii) 6 × 2 min post-dep DMF washes (9 ml/g resin), |
| | | (iii) 2.0 AA/2.2 DIC/2.0 Oxyma, in DMF (7.25 ml/g resin), rt., |
| | | (iv) 6 × 2 min, 10 volumes DMF (9 ml/g resin) post-coupling |
| | | washes |
| 15 | Fmoc- | (i) 3/4 × 30 min De-Fmoc cycles, |
| | Lys(ivDde)-OH | (ii) 6 × 2 min post-dep DMF washes (9 ml/g resin), |
| | | (iii) 8% hydrazine/DMF (9 ml/g resin), |
| | | (iv) 6 × 2 min, 10 volumes DMF (9 ml/g resin) post-coupling |
| | | washes |
| 16 | Fmoc-L-Ala-OH | (i) 3/4 × 30 min De-Fmoc cycles, |
| | | (ii) 6 × 2 min post-dep DMF washes (9 ml/g resin), |
| | | (iii) 2.0 AA/2.2 DIC/2.0 Oxyma, in DMF (7.25 ml/g resin), rt., |
| | | (iv) 6 × 2 min, 10 volumes DMF (9 ml/g resin) post-coupling |
| | | washes |
| 17 | Fmoc-L- | (i) 3/4 × 30 min De-Fmoc cycles, |
| | Lys(Boc)-OH | (ii) 6 × 2 min post-dep DMF washes (9 ml/g resin), |
| | | (iii) 2.0 AA/2.2 DIC/2.0 Oxyma, in DMF (7.25 ml/g resin), rt., |
| | | (iv) 6 × 2 min, 10 volumes DMF (9 ml/g resin) post-coupling |
| | | washes |
| 18 | Fmoc-L- | (i) 3/4 × 30 min De-Fmoc cycles, |
| | Lys(Boc)-OH | (ii) 6 × 2 min post-dep DMF washes (9 ml/g resin), |
| | | (iii) 2.0 AA/2.2 DIC/2.0 Oxyma, in DMF (7.25 ml/g resin), rt., |
| | | (iv) 6 × 2 min, 10 volumes DMF (9 ml/g resin) post-coupling |
| | | washes |
| 19 | Fmoc-L- | (i) 3/4 × 30 min De-Fmoc cycles, |
| | Glu(O^{t}Bu)-OH | (ii) 6 × 2 min post-dep DMF washes (9 ml/g resin), |
| | | (iii) 2.0 AA/2.2 DIC/2.0 Oxyma, in DMF (7.25 ml/g resin), rt., |
| | | (iv) 6 × 2 min, 10 volumes DMF (9 ml/g resin) post-coupling |
| | | washes |
| 20 | Fmoc-L- | (i) 3/4 × 30 min De-Fmoc cycles, |
| | Asp(O^{t}Bu)-OH | (ii) 6 × 2 min post-dep DMF washes (9 ml/g resin), |
| | | (iii) 2.0 AA/2.2 DIC/2.0 Oxyma, in DMF (7.25 ml/g resin), rt., |
| | | (iv) 6 × 2 min, 10 volumes DMF (9 ml/g resin) post-coupling |
| | | washes |
| 21 | Fmoc-L-Leu-OH | (i) 3/4 × 30 min De-Fmoc cycles, |
| | | (ii) 6 × 2 min post-dep DMF washes (9 ml/g resin), |
| | | (iii) 2.0 AA/2.2 DIC/2.0 Oxyma, in DMF (7.25 ml/g resin), rt., |
| | | (iv) 6 × 2 min, 10 volumes DMF (9 ml/g resin) post-coupling |
| | | washes |
| 22 | Fmoc-L- | (i) 3/4 × 30 min De-Fmoc cycles, |
| | Tyr(^{t}Bu)-OH | (ii) 6 × 2 min post-dep DMF washes (9 ml/g resin), |
| | | (iii) 2.0 AA/2.2 DIC/2.0 Oxyma, in DMF (7.25 ml/g resin), rt., |
| | | (iv) 6 × 2 min, 10 volumes DMF (9 ml/g resin) post-coupling |
| | | washes |
| 23 | Fmoc-L- | (i) 3/4 × 30 min De-Fmoc cycles, |
| | Lys(Boc)-OH | (ii) 6 × 2 min post-dep DMF washes (9 ml/g resin), |
| | | (iii) 2.0 AA/2.2 DIC/2.0 Oxyma, in DMF (7.25 ml/g resin), rt., |
| | | (iv) 6 × 2 min, 10 volumes DMF (9 ml/g resin) post-coupling |
| | | washes |
| 24 | Fmoc-L- | (i) 3/4 × 30 min De-Fmoc cycles, |
| | Ser(^{t}Bu)-OH | (ii) 6 × 2 min post-dep DMF washes (9 ml/g resin), |
| | | (iii) 2.0 AA/2.2 DIC/2.0 Oxyma, in DMF (7.25 ml/g resin), rt., |
| | | (iv) 6 × 2 min, 10 volumes DMF (9 ml/g resin) post-coupling |
| | | washes |
| 25 | Fmoc-L- | (i) 3/4 × 30 min De-Fmoc cycles, |
| | Tyr(^{t}Bu)-OH | (ii) 6 × 2 min post-dep DMF washes (9 ml/g resin), |
| | | (iii) 2.0 AA/2.2 DIC/2.0 Oxyma, in DMF (7.25 ml/g resin), rt., |
| | | (iv) 6 × 2 min, 10 volumes DMF (9 ml/g resin) post-coupling |
| | | washes |
| 26 | Fmoc-L- | (i) 3/4 × 30 min De-Fmoc cycles, |
| | Asp(O^{t}Bu)-OH | (ii) 6 × 2 min post-dep DMF washes (9 ml/g resin), |
| | | (iii) 2.0 AA/2.2 DIC/2.0 Oxyma, in DMF (7.25 ml/g resin), rt., |
| | | (iv) 6 × 2 min, 10 volumes DMF (9 ml/g resin) post-coupling |
| | | washes |
| 27 | Fmoc-L- | (i) 3/4 × 30 min De-Fmoc cycles, |
| | Ser(^{t}Bu)-OH | (ii) 6 × 2 min post-dep DMF washes (9 ml/g resin), |
| | | (iii) 2.0 AA/2.2 DIC/2.0 Oxyma, in DMF (7.25 ml/g resin), rt., |
| | | (iv) 6 × 2 min, 10 volumes DMF (9 ml/g resin) post-coupling |
| | | washes |
| 28 | Fmoc-L- | (i) 3/4 × 30 min De-Fmoc cycles, |
| | Thr(^{t}Bu)-OH | (ii) 6 × 2 min post-dep DMF washes (9 ml/g resin), |
| | | (iii) 2.0 AA/2.2 DIC/2.0 Oxyma, in DMF (7.25 ml/g resin), rt., |
| | | (iv) 6 × 2 min, 10 volumes DMF (9 ml/g resin) post-coupling |
| | | washes |
| 29 | Fmoc-L-Phe-OH | (i) 3/4 × 30 min De-Fmoc cycles, |
| | | (ii) 6 × 2 min post-dep DMF washes (9 ml/g resin), |
| | | (iii) 2.0 AA/2.2 DIC/2.0 Oxyma, in DMF (7.25 ml/g resin), rt., |
| | | (iv) 6 × 2 min, 10 volumes DMF (9 ml/g resin) post-coupling |
| | | washes |

### Fmoc Deprotection:

Resin in the peptide reactor is treated with either three or four charges of the 20% v/v Pip/DMF solution. Each treatment is stirred on the resin for 30 min followed by filtration to complete Fmoc protecting group removal. After the final 20% v/v PIP/DMF treatment, the resin bed is washed a minimum of six times with DMF at the pre-specified DMF volume charge.

### Amino Acid Activation:

A pre-prepared solution of 12% w/w Oxyma Pure/DMF is charged to a reactor. The selected Fmoc amino acid is then added. The mixture is stirred at 20±5° C. until the Fmoc amino acid has completely dissolved. The Fmoc-AA/Oxyma Pure/DMF solutions are then cooled to 15±3° C. prior to activation to ensure the minor exothermic activation reaction is controlled and the resulting solution temperature is maintained in the range specified of 20±5° C. The amino acid solution is activated by DIC addition. The activated ester solution is stirred for 20-30 minutes prior to transfer of the solution to the reactor containing the peptide on resin compound.

### Coupling:

Upon completion of the activation step, the activated ester solution is transferred to the reactor containing deprotected peptide on resin to initiate the coupling reaction. The peptide coupling reaction is stirred at 20±5° C. for at least 4 hours. After the required stir time, the resin slurry is sampled for coupling completion (IPC). Sampling is repeated at specific intervals as needed until a passing IPC result is obtained. Re-coupling operations are performed, if necessary. When the coupling is complete, the peptide reactor solution contents are filtered then the peptide on resin compounds are washed several times with DMF to prepare for the next coupling.

### Example 3: Preparation of the Boc-His(Dnp)-Aib-Gln(Trt)-Gly-Thr(^{t}Bu)-OH Pentamer (SEQ ID NO: 14)

### Synthesis of Preparation 6

Boc-His(Dnp)-Aib-Gln(Trt)-Gly-Thr(^{t}Bu)-OH SEQ ID NO: 14

### Resin Charging:

Reactors 1-3 are each charged with one-third of the amount of Fmoc-L-Thr(tBu)-OH on CTC resin (0.769 mmol/g, 100-200 mesh, 2.94 g, 2.26 mmol). The resin is swelled with 3×15 ml of DMF for 20 minutes each, deprotected with 3×15 ml of 20% Pip/DMF for 30 minutes each, and washed with 5×15 ml of DMF for 1 minute each prior to the first coupling.

### Fmoc-Gly-OH Coupling:

A solution is prepared of 2-(9H-fluoren-9-ylmethoxycarbonylamino)acetic acid (2.01 g, 6.76 mmol) and ethyl cyanoglyoxylate-2-oxime (960 mg, 6.688 mmol) in 40.5 ml of DMF in a 60 ml bottle. N,N′-di-isopropylcarbodiimide (1.17 mL, 7.47 mmol) is added to this light yellow solution and the orange-yellow solution is allowed to stand for 30 minutes with occasional shaking. One-third of the solution is added by pipette directly to each reactor and the reaction is mixed for 12 hours and drained. The resin is washed with 5×15 ml of DMF for 1 minute each, deprotected with 4×15 ml of 20% Pip/DMF (v/v) for 30 minutes each, and then washed with 5×15 ml of DMF for 1 minute each and taken to the next coupling.

### Fmoc-L-Gln(Trt)-OH Coupling:

A solution is prepared of (2S)-2-(9H-fluoren-9-ylmethoxycarbonylamino)-5-oxo-5-(tritylamino)pentanoic acid (4.12 g, 6.75 mmol) and ethyl cyanoglyoxylate-2-oxime (960 mg, 6.688 mmol) in 40.5 ml of DMF in a 60 ml bottle. N,N′-di-isopropylcarbodiimide (1.17 mL, 7.47 mmol) is added to this light yellow solution and the orange-yellow solution is allowed to stand for 30 minutes with occasional shaking. One-third of the solution is added by pipette directly to each reactor and the reaction is mixed for 12 hours and then drained. The resin is washed with 5×15 ml of DMF for 1 minute each, deprotected with 4×15 ml of 20% Pip/DMF (v/v) for 30 minutes each, and then washed with 5×15 ml of DMF for 1 minute each and taken to the next coupling.

### Fmoc-Aib-OH Coupling:

A solution is prepared of 2-(9H-fluoren-9-ylmethoxycarbonylamino)-2-methyl-propanoic acid (2.20 g, 6.76 mmol) and ethyl cyanoglyoxylate-2-oxime (960 mg, 6.688 mmol) in 40.5 ml of DMF in a 60 ml bottle. N,N′-di-isopropylcarbodiimide (1.17 mL, 7.47 mmol) is added to this light yellow solution and the orange-yellow solution is allowed to stand for 30 minutes with occasional shaking. One-third of the solution is added by pipette directly to each reactor and the reaction is mixed for 18 hours and drained. The resin is washed with 5×15 ml of DMF for 1 minute each, deprotected with 4×15 ml of 20% Pip/DMF (v/v) for 30 minutes each, and then washed with 5×15 ml of DMF for 1 minute each and taken to the next coupling.

### Boc-L-his(Dnp)-OH Coupling:

A solution is prepared of Boc-His(dnp)-OH (2.84 g, 6.74 mmol) and ethyl cyanoglyoxylate-2-oxime (960 mg, 6.688 mmol) in 40.5 ml of DMF in a 60 ml bottle. N,N′-di-isopropylcarbodiimide (1.17 mL, 7.47 mmol) is added to this bright yellow solution and one-third of the orange-yellow solution is added immediately to each reactor. The reaction is mixed for 18 hours and then drained. The resin is washed with 5×15 ml of DMF for 1 minute each, 5×15 ml of DCM for 1 minute each, then drain dried for 4 hours.

Cleavage from Resin:

The combined peptide on resin is divided into two portions and each portion is suspended in 30 ml of 30% hexafluoroisopropanol (HFIP)/DCM (v/v) in a 40 ml reaction vial and mixed on a rotary mixer for 2 hours. The resins are filtered off on a fritted filter and washed in two portions with a total of 30 ml of DCM. The combined filtrate and washes are concentrated to a yellow dry foam by rotovap and then triturated twice with methyl tert-butyl ether (MTBE), each time concentrating to dryness on the rotovap (to remove HFIP), to give a bright yellow-orange powdery solid. The solid is triturated with 50 ml of cold 1:1 MTBE/heptane and sonicated, which produced a yellow suspension. The suspension is transferred to a centrifuge tube and centrifuged. The solid may not settle very well into a pellet, so another 30 ml of cold MTBE/heptane is added and the solid is filtered on a Buchner funnel, washed with a small amount of cold 1:1 MTBE/heptane, and dried overnight in the vacuum oven at 35° C. to give 2.255 g (91.4%) of a yellow solid with a UPLC purity of 88.1%.

### Example 4: Preparation of the Compound of SEQ ID NO: 12

### Synthesis of Preparation 7

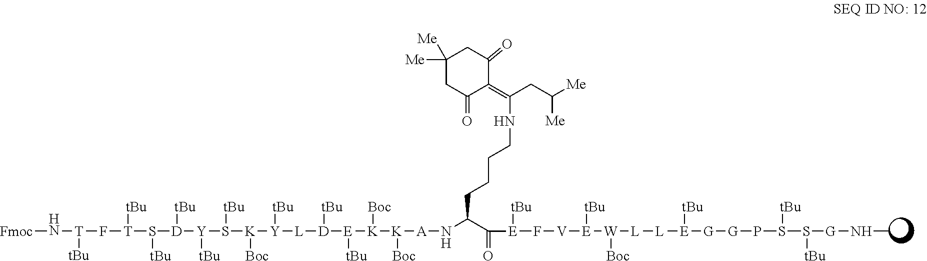

A Fmoc Sieber resin (0.6-0.8 mmol/g) is charged to a reactor is swelled with DMF, stirred for 2 hours, then DMF filtered off from the resin. The resin is then washed with DMF for a total of two times. The Fmoc-protected resin is then de-protected using 20% Pip/DMF treatments at 9 ml/g resin. Sampling to verify Fmoc removal is performed after the last Pip/DMF treatment to confirm >99% Fmoc removal via UV analysis (IPC target <1% Fmoc remaining). After the final 20% w/w Pip/DMF treatment, the resin bed is washed multiple times with DMF (e.g. 6×2 min, 10 volume DMF washes at 9 ml/g resin). The peptide backbone is built out using the following conditions for each amino acid coupling and deprotection:

| Cycle | Amino acid | SPPS conditions |
|---|---|---|
| 1 | Fmoc-L-Gly-OH | (i) 3/4 × 30 min De-Fmoc cycles, |
| | | (ii) 6 × 2 min post-dep DMF washes (9 ml/g resin), |
| | | (iii) 2.0 AA/2.2 DIC/2.0 Oxyma, in DMF (7.25 ml/g resin), (room |
| | | temperature (rt), |
| | | (iv) 6 × 2 min, 10 volumes DMF (9 ml/g resin) post-coupling |
| | | washes |
| 2 | Fmoc-L- | (i) 3/4 × 30 min De-Fmoc cycles, |
| | Ser(^{t}Bu)-OH | (ii) 6 × 2 min post-dep DMF washes (9 ml/g resin), |
| | | (iii) 2.0 AA/2.2 DIC/2.0 Oxyma, in DMF (7.25 ml/g resin), rt., |
| | | (iv) 6 × 2 min, 10 volumes DMF (9 ml/g resin) post-coupling |
| | | washes |
| 3 | Fmoc-L- | (i) 3/4 × 30 min De-Fmoc cycles, |
| | Ser(^{t}Bu)-OH; | (ii) 6 × 2 min post-dep DMF washes (9 ml/g resin), |
| | | (iii) 2.0 AA/2.2 DIC/2.0 Oxyma, in DMF (7.25 ml/g resin), rt., |
| | | (iv) 6 × 2 min, 10 volumes DMF (9 ml/g resin) post-coupling |
| | | washes |
| 4 | Fmoc-L-Pro-OH | (i) 3/4 × 30 min De-Fmoc cycles, |
| | | (ii) 6 × 2 min post-dep DMF washes (9 ml/g resin), |
| | | (iii) 2.0 AA/2.2 DIC/2.0 Oxyma, in DMF (7.25 ml/g resin), rt., |
| | | (iv) 6 × 2 min, 10 volumes DMF (9 ml/g resin) post-coupling |
| | | washes |
| 5 | Fmoc-L-Gly-OH | (i) 3/4 × 30 min De-Fmoc cycles, |
| | | (ii) 6 × 2 min post-dep DMF washes (9 ml/g resin), |
| | | (iii) 2.0 AA/2.2 DIC/2.0 Oxyma, in DMF (7.25 ml/g resin), rt., |
| | | (iv) 6 × 2 min, 10 volumes DMF (9 ml/g resin) post-coupling |
| | | washes |
| 6 | Fmoc-L-Gly-OH | (i) 3/4 × 30 min De-Fmoc cycles, |
| | | (ii) 6 × 2 min post-dep DMF washes (9 ml/g resin), |
| | | (iii) 2.0 AA/2.2 DIC/2.0 Oxyma, in DMF (7.25 ml/g resin), rt., |
| | | (iv) 6 × 2 min, 10 volumes DMF (9 ml/g resin) post-coupling |
| | | washes |
| 7 | Fmoc-L- | (i) 3/4 × 30 min De-Fmoc cycles, |
| | Glu(O^{t}Bu)-OH | (ii) 6 × 2 min post-dep DMF washes (9 ml/g resin), |
| | | (iii) 2.0 AA/2.2 DIC/2.0 Oxyma, in DMF (7.25 ml/g resin), rt., |
| | | (iv) 6 × 2 min, 10 volumes DMF (9 ml/g resin) post-coupling |
| | | washes |
| 8 | Fmoc-L-Leu-OH | (i) 3/4 × 30 min De-Fmoc cycles, |
| | | (ii) 6 × 2 min post-dep DMF washes (9 ml/g resin), |
| | | (iii) 2.0 AA/2.2 DIC/2.0 Oxyma, in DMF (7.25 ml/g resin), rt., |
| | | (iv) 6 × 2 min, 10 volumes DMF (9 ml/g resin) post-coupling |
| | | washes |
| 9 | Fmoc-L-Leu-OH | (i) 3/4 × 30 min De-Fmoc cycles, |
| | | (ii) 6 × 2 min post-dep DMF washes (9 ml/g resin), |
| | | (iii) 2.0 AA/2.2 DIC/2.0 Oxyma, in DMF (7.25 ml/g resin), rt., |
| | | (iv) 5 × 2 min, 10 volumes DMF (9 ml/g resin) post-coupling |
| | | washes |
| 10 | Fmoc-L- | (i) 3/4 × 30 min De-Fmoc cycles, |
| | Trp(Boc)-OH | (ii) 6 × 2 min post-dep DMF washes (9 ml/g resin), |
| | | (iii) 2.0 AA/2.2 DIC/2.0 Oxyma, in DMF (7.25 ml/g resin), rt., |
| | | (iv) 6 × 2 min, 10 volumes DMF (9 ml/g resin) post-coupling |
| | | washes |
| 11 | Fmoc-L- | (i) 3/4 × 30 min De-Fmoc cycles, |
| | Glu(O^{t}Bu)-OH | (ii) 6 × 2 min post-dep DMF washes (9 ml/g resin), |
| | | (iii) 2.0 AA/2.2 DIC/2.0 Oxyma, in DMF (7.25 ml/g resin), rt., |
| | | (iv) 6 × 2 min, 10 volumes DMF (9 ml/g resin) post-coupling |
| | | washes |
| 12 | Fmoc-L-Val-OH | (i) 3/4 × 30 min De-Fmoc cycles, |
| | | (ii) 6 × 2 min post-dep DMF washes (9 ml/g resin), |
| | | (iii) 2.0 AA/2.2 DIC/2.0 Oxyma, in DMF (7.25 ml/g resin), rt., |
| | | (iv) 6 × 2 min, 10 volumes DMF (9 ml/g resin) post-coupling |
| | | washes |
| 13 | Fmoc-L-Phe-OH | (i) 3/4 × 30 min De-Fmoc cycles, |
| | | (ii) 6 × 2 min post-dep DMF washes (9 ml/g resin), |
| | | (iii) 2.0 AA/2.2 DIC/2.0 Oxyma, in DMF (7.25 ml/g resin), rt., |
| | | (iv) 6 × 2 min, 10 volumes DMF (9 ml/g resin) post-coupling |
| | | washes |
| 14 | Fmoc-L- | (i) 3/4 × 30 min De-Fmoc cycles, |
| | Glu(O^{t}Bu)-OH | (ii) 6 × 2 min post-dep DMF washes (9 ml/g resin), |
| | | (iii) 2.0 AA/2.2 DIC/2.0 Oxyma, in DMF (7.25 ml/g resin), rt., |
| | | (iv) 6 × 2 min, 10 volumes DMF (9 ml/g resin) post-coupling |
| | | washes |
| 15 | Fmoc- | (i) 3/4 × 30 min De-Fmoc cycles, |
| | Lys(ivDde)-OH | (ii) 6 × 2 min post-dep DMF washes (9 ml/g resin), |
| | | (iii) 8% hydrazine/DMF (9 ml/g resin), |
| | | (iv) 6 × 2 min, 10 volumes DMF (9 ml/g resin) post-coupling |
| | | washes |
| 16 | Fmoc-L-Ala-OH | (i) 3/4 × 30 min De-Fmoc cycles, |
| | | (ii) 6 × 2 min post-dep DMF washes (9 ml/g resin), |
| | | (iii) 2.0 AA/2.2 DIC/2.0 Oxyma, in DMF (7.25 ml/g resin), rt., |
| | | (iv) 6 × 2 min, 10 volumes DMF (9 ml/g resin) post-coupling |
| | | washes |
| 17 | Fmoc-L- | (i) 3/4 × 30 min De-Fmoc cycles, |
| | Lys(Boc)-OH | (ii) 6 × 2 min post-dep DMF washes (9 ml/g resin), |
| | | (iii) 2.0 AA/2.2 DIC/2.0 Oxyma, in DMF (7.25 ml/g resin), rt., |
| | | (iv) 6 × 2 min, 10 volumes DMF (9 ml/g resin) post-coupling |
| | | washes |
| 18 | Fmoc-L- | (i) 3/4 × 30 min De-Fmoc cycles, |
| | Lys(Boc)-OH | (ii) 6 × 2 min post-dep DMF washes (9 ml/g resin), |
| | | (iii) 2.0 AA/2.2 DIC/2.0 Oxyma, in DMF (7.25 ml/g resin), rt., |
| | | (iv) 6 × 2 min, 10 volumes DMF (9 ml/g resin) post-coupling |
| | | washes |
| 19 | Fmoc-L- | (i) 3/4 × 30 min De-Fmoc cycles, |
| | Glu(O^{t}Bu)-OH | (ii) 6 × 2 min post-dep DMF washes (9 ml/g resin), |
| | | (iii) 2.0 AA/2.2 DIC/2.0 Oxyma, in DMF (7.25 ml/g resin), rt., |
| | | (iv) 6 × 2 min, 10 volumes DMF (9 ml/g resin) post-coupling |
| | | washes |
| 20 | Fmoc-L- | (i) 3/4 × 30 min De-Fmoc cycles, |
| | Asp(O^{t}Bu)-OH | (ii) 6 × 2 min post-dep DMF washes (9 ml/g resin), |
| | | (iii) 2.0 AA/2.2 DIC/2.0 Oxyma, in DMF (7.25 ml/g resin), rt., |
| | | (iv) 6 × 2 min, 10 volumes DMF (9 ml/g resin) post-coupling |
| | | washes |
| 21 | Fmoc-L-Leu-OH | (i) 3/4 × 30 min De-Fmoc cycles, |
| | | (ii) 6 × 2 min post-dep DMF washes (9 ml/g resin), |
| | | (iii) 2.0 AA/2.2 DIC/2.0 Oxyma, in DMF (7.25 ml/g resin), rt., |
| | | (iv) 6 × 2 min, 10 volumes DMF (9 ml/g resin) post-coupling |
| | | washes |
| 22 | Fmoc-L- | (i) 3/4 × 30 min De-Fmoc cycles, |
| | Tyr(^{t}Bu)-OH | (ii) 6 × 2 min post-dep DMF washes (9 ml/g resin), |
| | | (iii) 2.0 AA/2.2 DIC/2.0 Oxyma, in DMF (7.25 ml/g resin), rt., |
| | | (iv) 6 × 2 min, 10 volumes DMF (9 ml/g resin) post-coupling |
| | | washes |
| 23 | Fmoc-L- | (i) 3/4 × 30 min De-Fmoc cycles, |
| | Lys(Boc)-OH | (ii) 6 × 2 min post-dep DMF washes (9 ml/g resin), |
| | | (iii) 2.0 AA/2.2 DIC/2.0 Oxyma, in DMF (7.25 ml/g resin), rt., |
| | | (iv) 6 × 2 min, 10 volumes DMF (9 ml/g resin) post-coupling |
| | | washes |
| 24 | Fmoc-L- | (i) 3/4 × 30 min De-Fmoc cycles, |
| | Ser(^{t}Bu)-OH | (ii) 6 × 2 min post-dep DMF washes (9 ml/g resin), |
| | | (iii) 2.0 AA/2.2 DIC/2.0 Oxyma, in DMF (7.25 ml/g resin), rt., |
| | | (iv) 6 × 2 min, 10 volumes DMF (9 ml/g resin) post-coupling |
| | | washes |
| 25 | Fmoc-L- | (i) 3/4 × 30 min De-Fmoc cycles, |
| | Tyr(^{t}Bu)-OH | (ii) 6 × 2 min post-dep DMF washes (9 ml/g resin), |
| | | (iii) 2.0 AA/2.2 DIC/2.0 Oxyma, in DMF (7.25 ml/g resin), rt., |
| | | (iv) 6 × 2 min, 10 volumes DMF (9 ml/g resin) post-coupling |
| | | washes |
| 26 | Fmoc-L- | (i) 3/4 × 30 min De-Fmoc cycles, |
| | Asp(O^{t}Bu)-OH | (ii) 6 × 2 min post-dep DMF washes (9 ml/g resin), |
| | | (iii) 2.0 AA/2.2 DIC/2.0 Oxyma, in DMF (7.25 ml/g resin), rt., |
| | | (iv) 6 × 2 min, 10 volumes DMF (9 ml/g resin) post-coupling |
| | | washes |
| 27 | Fmoc-L- | (i) 3/4 × 30 min De-Fmoc cycles, |
| | Ser(^{t}Bu)-OH | (ii) 6 × 2 min post-dep DMF washes (9 ml/g resin), |
| | | (iii) 2.0 AA/2.2 DIC/2.0 Oxyma, in DMF (7.25 ml/g resin), rt., |
| | | (iv) 6 × 2 min, 10 volumes DMF (9 ml/g resin) post-coupling |
| | | washes |
| 28 | Fmoc-L- | (i) 3/4 × 30 min De-Fmoc cycles, |
| | Thr(^{t}Bu)-OH | (ii) 6 × 2 min post-dep DMF washes (9 ml/g resin), |
| | | (iii) 2.0 AA/2.2 DIC/2.0 Oxyma, in DMF (7.25 ml/g resin), rt., |
| | | (iv) 6 × 2 min, 10 volumes DMF (9 ml/g resin) post-coupling |
| | | washes |
| 29 | Fmoc-L-Phe-OH | (i) 3/4 × 30 min De-Fmoc cycles, |
| | | (ii) 6 × 2 min post-dep DMF washes (9 ml/g resin), |
| | | (iii) 2.0 AA/2.2 DIC/2.0 Oxyma, in DMF (7.25 ml/g resin), rt., |
| | | (iv) 6 × 2 min, 10 volumes DMF (9 ml/g resin) post-coupling |
| | | washes |
| 30 | Fmoc-L- | (i) 3/4 × 30 min De-Fmoc cycles, |
| | Thr(^{t}Bu)-OH | (ii) 6 × 2 min post-dep DMF washes (9 ml/g resin), |
| | | (iii) 2.0 AA/2.2 DIC/2.0 Oxyma, in DMF (7.25 ml/g resin), rt., |
| | | (iv) 6 × 2 min, 10 volumes DMF (9 ml/g resin) post-coupling |
| | | washes |

### Fmoc Deprotection:

Resin in the peptide reactor is treated with either three or four charges of the 20% v/v Pip/DMF solution. Each treatment is stirred on the resin for 30 min followed by filtration to complete Fmoc protecting group removal. After the final 20% v/v PIP/DMF treatment, the resin bed is washed a minimum of six times with DMF at the pre-specified DMF volume charge.

### Amino Acid Activation:

A pre-prepared solution of 12% w/w Oxyma Pure/DMF is charged to a reactor. The selected Fmoc amino acid is then added. The mixture is stirred at 20±5° C. until the Fmoc amino acid has completely dissolved. The Fmoc-AA/Oxyma Pure/DMF solutions are then cooled to 15±3° C. prior to activation to ensure the minor exothermic activation reaction is controlled and the resulting solution temperature is maintained in the range specified of 20±5° C. The amino acid solution is activated by DIC addition. The activated ester solution is stirred for 20-30 minutes prior to transfer of the solution to the reactor containing the peptide on resin compound.

### Coupling:

Upon completion of the activation step, the activated ester solution is transferred to the reactor containing deprotected peptide on resin to initiate the coupling reaction. The peptide coupling reaction is stirred at 20±5° C. for at least 4 hours. After the required stir time, the resin slurry is sampled for coupling completion (IPC). Sampling is repeated at specific intervals as needed until a passing IPC result is obtained. Re-coupling operations are performed, if necessary. When the coupling is complete, the peptide reactor solution contents are filtered then the peptide on resin compounds are washed several times with DMF to prepare for the next coupling.

### Example 5: Preparation of the Compound of SEQ ID NO: 16

### Synthesis of Preparation 8

Boc-His(Dnp)-Aib-Gln(Trt)-Gly-OH SEQ ID NO: 16

### Resin Charging:

Three separate bottom-fritted reactors are each charged one-third of Fmoc-Gly-OH on CTC resin (100-200 mesh, 2.98 g, 2.25 mmol, 0.756 mmol/g loading). Each resin is swelled with 3×15 ml of DMF for 20 minutes each, Fmoc-deprotected with 3×15 ml of 20% piperidine/DMF (v/v) for 30 minutes and washed with 5×15 ml of DMF for 1 minute each prior to the first coupling.

### Fmoc-Gln(Trt)-OH Coupling:

A solution is prepared of (2S)-2-(9H-fluoren-9-ylmethoxycarbonylamino)-5-oxo-5-(tritylamino)pentanoic acid (4.12 g, 6.75 mmol) and ethyl cyanoglyoxylate-2-oxime (969.0 mg, 6.750 mmol) in 40.5 ml of DMF in a 60 ml bottle. N,N′-di-isopropylcarbodiimide (937.0 mg, 7.425 mmol, 100 mass %) is added to this light yellow solution and the orange-yellow solution is allowed to stand for 30 minutes with occasional shaking. One-third of the solution is added by pipette directly to each reactor and the reaction is mixed for 12 hours and then drained. The resin is washed with 5×15 ml of DMF for 1 minute each, deprotected with 4×15 ml of 20% Pip/DMF (v/v) for 30 minutes each, and then washed with 5×15 ml of DMF for 1 minute each and taken directly to the next coupling.

### Fmoc-Aib-OH Coupling:

A solution is prepared of 2-(9H-fluoren-9-ylmethoxycarbonylamino)-2-methyl-propanoic acid (J, 2.20 g, 6.76 mmol) and ethyl cyanoglyoxylate-2-oxime (969.0 mg, 6.750 mmol) in 40.5 ml of DMF in a 60 ml bottle. N,N′-di-isopropylcarbodiimide (937.0 mg, 7.425 mmol) is added to this light yellow solution and the orange-yellow solution is allowed to stand for 30 minutes with occasional shaking. One-third of the solution is added by pipette directly to each reactor and the reaction is mixed for 18 hours and then drained. The resin is washed with 5×15 ml of DMF for 1 minute each, deprotected with 4×15 ml of 20% Pip/DMF (v/v) for 30 minutes each, and then washed with 5×15 ml of DMF for 1 minute each and taken to the next coupling.

### Boc-his(Dnp)-OH Coupling:

A solution is prepared of Boc-His(Dnp)-OH (D, 2.84 g, 6.74 mmol) and ethyl cyanoglyoxylate-2-oxime (969.0 mg, 6.750 mmol) in 40.5 ml of DMF in a 60 ml bottle. N,N′-di-isopropylcarbodiimide (937.0 mg, 7.425 mmol) is added to this bright yellow solution and one-third of the orange-yellow solution is added immediately to each reactor. The reaction is mixed for 18 hours and then drained. The resin is washed with 5×15 ml of DMF for 1 minute each, 5×15 ml of DCM for 1 minute each, then drain dried for 4 hours.

Cleavage of the Peptide from Resin:

The combined peptide on resin from all three reactors is divided into two portions and each portion is suspended in 30 ml of 30% hexafluoroisopropanol (HFIP)/DCM (v/v) in a 40 ml reaction vial and mixed on a rotary mixer for 2 hours. The resins are filtered off on a fritted funnel and washed in two portions with a total of 30 ml of DCM. The combined filtrate and washes are concentrated to a yellow dry foam by rotovap and then triturated twice with methyl tert-butyl ether (MTBE), each time concentrating to dryness on the rotovap (to remove residual HFIP), to give a bright yellow-orange powdery solid. The solid is triturated with 50 ml of 1:1 MTBE/heptane and sonicated, which produced a nice yellow suspension. The suspension is transferred to a centrifuge tube and centrifuged. After decanting the supernatant, the solid is washed twice in the same way with 30 ml of MTBE and, after partially drying with a stream of nitrogen, the solid is dried overnight in the vacuum oven at 35° C. to give 1.89 g (87.8%) of a yellow solid with 97.66% UPLC purity.

### Example 6: Preparation of ^{t}BuO-C20-γGlu(^{t}Bu)-AEEA-AEEA-OH

### Synthesis of Preparation 9

### (3,6,12,15-Tetraoxa-9,18-diazatricosanedioic acid, 22-[[20-(1,1-dimethylethoxy)-1,20-dioxoeicosyl]amino]-10,19-dioxo-, 2,3-(1,1-dimethylethyl) ester, (22S))

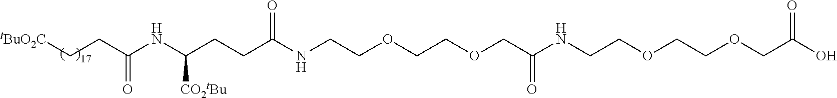

The synthesis is conducted with an automated peptide synthesizer.

### Solvent and Reagent Preparation:

Twenty (20) L DMF is charged to the solvent reservoir.

Four (4) L of 20% Pip/DMF solution is charged to the piperidine reservoir.

444 mL of 0.4 M HATU solution is prepared using HATU (67.53 g, 177.6 mmol, 100 mass %) and DMF, then charged to the appropriate solvent bottle.

444 mL of 1.0 M DIEA solution is prepared using N,N-diisopropylethylamine (77.55 mL, 445 mmol, 100 mass %) and DMF, and subsequently charged to the appropriate solvent bottle.

Four (4) L of CH₂Cl₂ is charged to the DCM solvent bottle. 1 L of CH₂Cl₂ is charged to the second DCM solvent bottle.

### Amino Acid Solution Preparation:

137 mL of 0.400 M ^{t}BuO-C₂₀—OH solution is prepared from 20-tert-butoxy-20-oxo-icosanoic acid (21.843 g, 54.80 mmol, 100 mass %) and DMF/toluene mixture (1:1), then charged to the addition bottle.

137 mL of 0.400 M FmocNH-Glu-O^{t}Bu solution is prepared from (4R)-5-tert-butoxy-4-(9H-fluoren-9-ylmethoxycarbonylamino)-5-oxo-pentanoic acid (23.316 g, 54.80 mmol, 100 mass %) and DMF, then charged to the addition bottle.

137 mL of 0.400 M FmocNH-AEEA-OH solution is prepared from 2-[2-[2-(9H-fluoren-9-ylmethoxycarbonylamino)ethoxy]ethoxy]acetic acid (21.121 g, 54.80 mmol, 100 mass %) and DMF and then charged to the addition bottle.

The coupling conditions are as follows: 0.133 M, 2.0 equiv HATU, 5.0 equiv DIEA, ambient temperature, 3 hours, deprotection for 3×15 min with 20% piperidine/DMF.

### Resin Charging:

A 2-CTC resin (0.99 mmol/g) is used in this synthesis and is charged with FmocNH-AEEA] 1.01 g is added in each of twenty-four parallel reactions.

### Symphony X Automatic Program (Per 1.0 Mmol Scale Reaction):

### (i) Swell:

3×15 mL DMF for 10 min

### (ii) Cycle:

3×15 ml 20% Pip/DMF for 15 min each
5×15 mL DMF wash for 30 sec each
5 mL amino acid
5 mL DIEA
5 mL HATU
Stir for 3 hour
5×15 mL DMF wash for 30 sec each
   (iii) Dry:
5×15 mL methylene chloride for 30 sec each
Drain dry for 2 h

### Cleavage Protocol:

The resin is cleaved by stirring the combined lots in 30% HFIP/CH₂Cl₂ (240 mL) for 1.5 hours. The resin is filtered, washed with additional CH₂Cl₂ (2×50 mL) and the solvent is removed from the filtrate in vacuo. The resulting oil is redissolved in acetonitrile and solvent is removed again. This operation is repeated to provide 30.47 g (146% of theoretical yield) of a viscous yellow oil, which contained 52.3 area % desired product by UPLC analysis.

### Chromatography:

The crude product (30.47 g, 52.3 area % purity) is purified by flash chromatography (500 grams of silica gel, eluted with 85% dichloromethane/10% methanol/5% acetic acid, 38×100 ml fractions collected). The desired product elutes in fractions 17-34, with a few mixed fractions before and after the clean product being discarded. Fractions 17-34 are concentrated under reduced pressure to a light yellow viscous liquid and then the residual acetic acid is removed by azeotropic distillation under reduced pressure twice with heptane to yield 17.94 g of purified product as a light yellow viscous oil with 86.6 HPLC area % purity.

### Crystallization:

The chromatography concentrate (17.94 g) is taken up in 120 ml of acetonitrile in a 250 ml Erlenmeyer flask and the mixture is stirred for about 10 minutes at ambient temperature until a light yellow solution had formed. The solution is cooled for about 4 hours at −20 to −25° C. Significant solid precipitates and is especially thick on the inside surfaces of the flask. A spatula is used to break up the solid, which yields a well-dispersed suspension. The solid is kept at −20 to −25° C. and a fritted glass filter and acetonitrile for the wash are pre-cooled to −20 to −25° C. in the freezer. The suspension is filtered quickly and washed with approximately 50 ml of the cold acetonitrile. The solid is quickly scraped off the filter and transferred to a glass bottle. The solid melts to a thick colorless oil, which solidifies upon cooling to −20° C. Total yield of preparation 9 is 13.4 g (74.7% yield), with a UPLC purity of 91.65 area %.

### SEQUENCES

H₂N-H-Aib-Q-G-T-F-T-S-D-Y-S-K-Y-L-D-E-K-K-A-K-E-F-V-E-W-L-L-E-G-G-P-S-S-G-NH₂ 1) SEQ ID NO: 1

wherein lysine (Lys/K) at position 20 is chemically modified by conjugation of the epsilon-amino group of the lysine side chain with ([2-(2-aminoethoxy)-ethoxy]-acetyl)₂-(γ-Glu)-CO—(CH₂)₁₈CO₂H

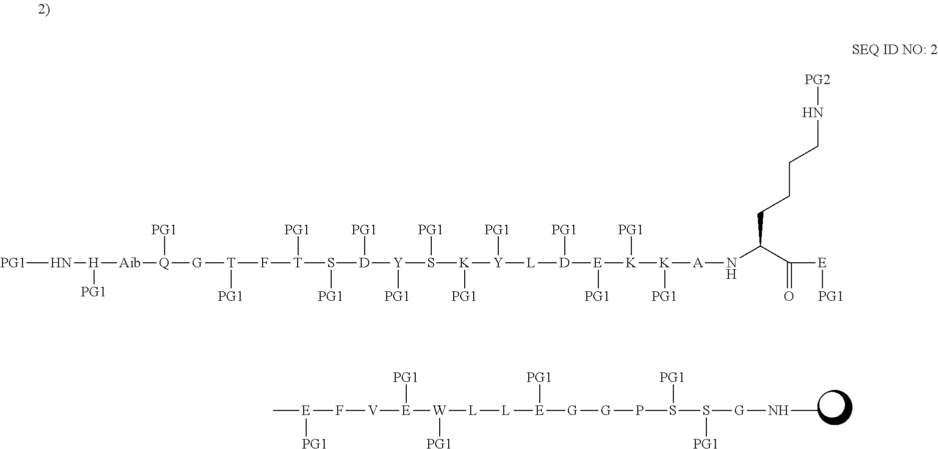

wherein PG1 is a base stable side-chain protecting group,
wherein Thr at position 5 is optionally protected with PG1
and wherein PG2 is an ivDde, Dde or Alloc side-chain protecting group

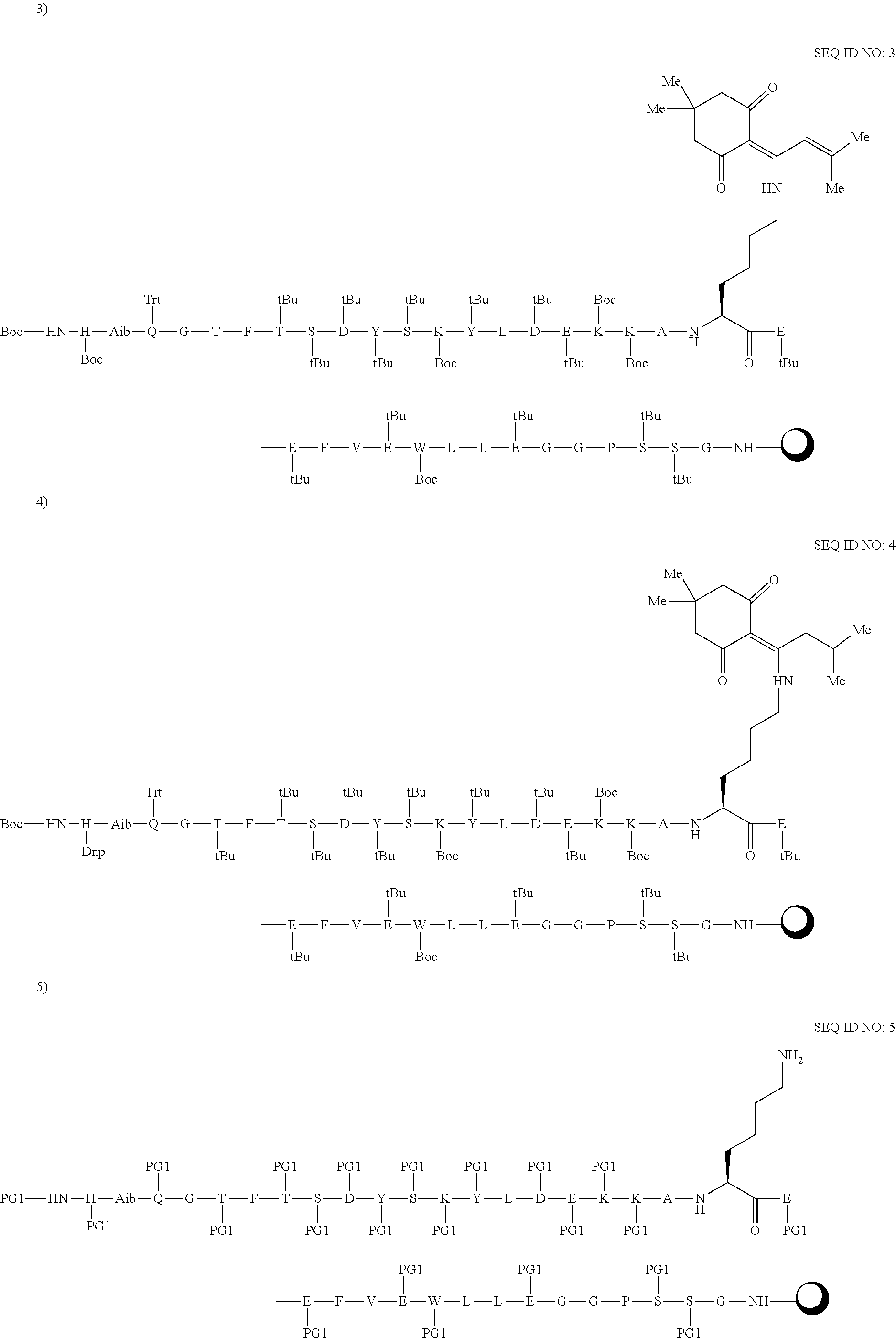

wherein PG1 is a base stable side-chain protecting group,
wherein Thr at position 5 is optionally protected with PG1

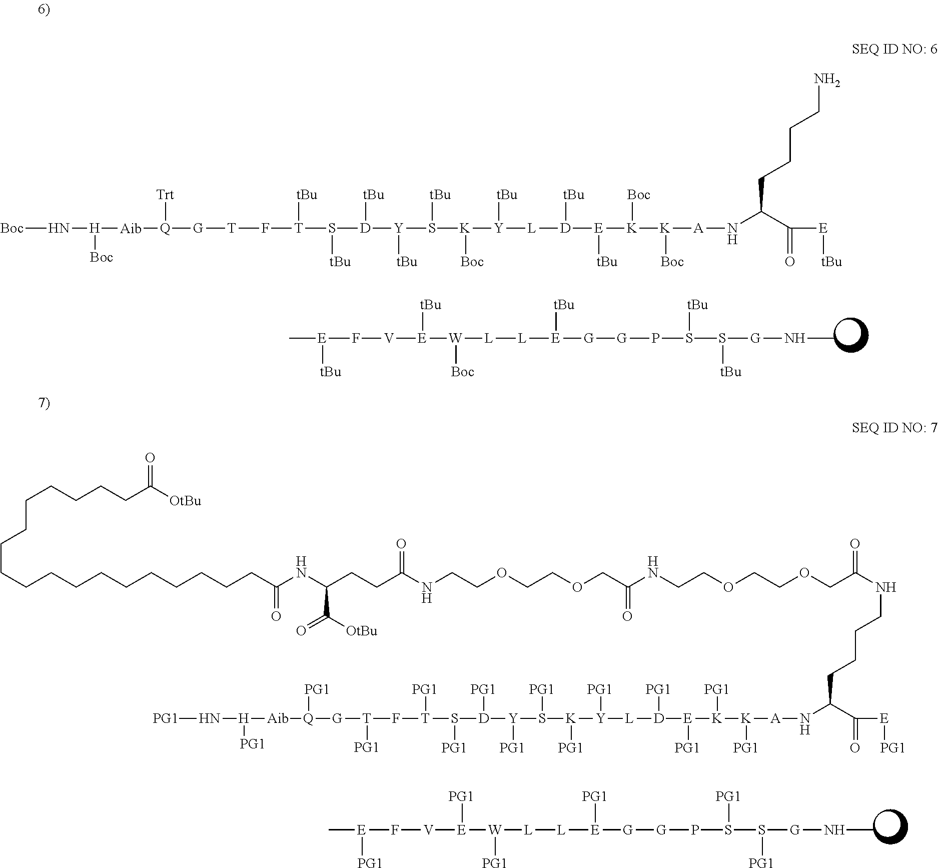

wherein PG1 is a base stable side-chain protecting group
wherein Thr at position 5 is optionally protected with PG1

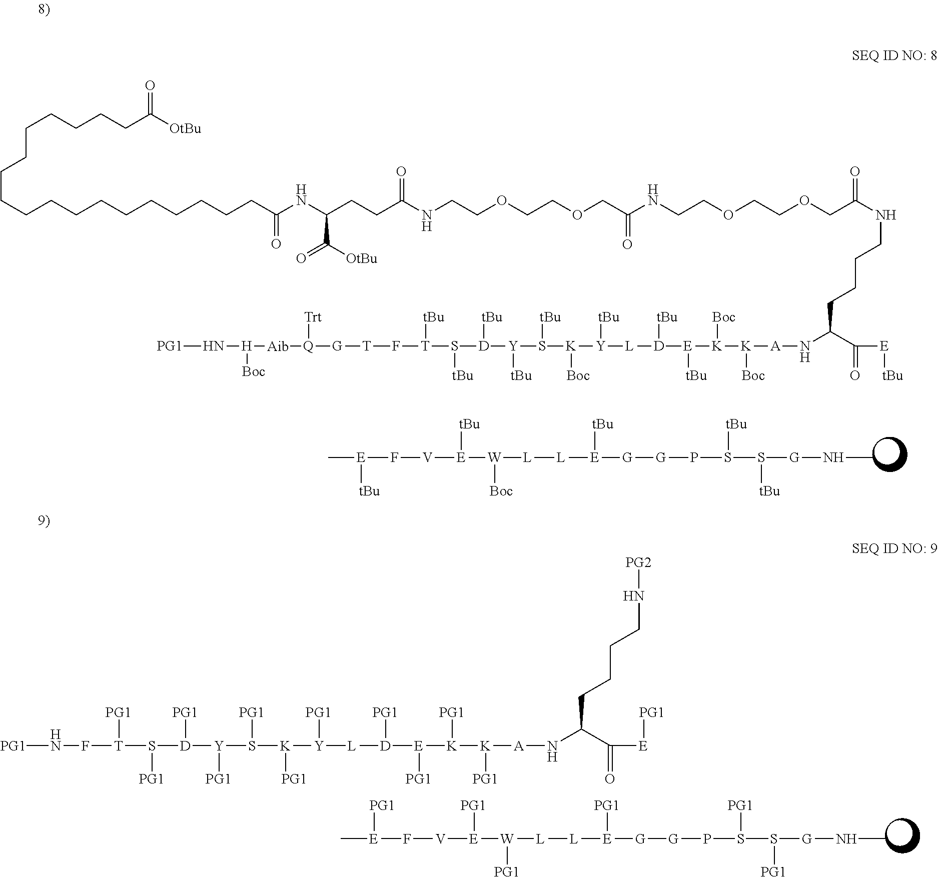

wherein PG1 is a base stable side-chain protecting group,
wherein PG2 is an ivDde, Dde or Alloc side-chain protecting group

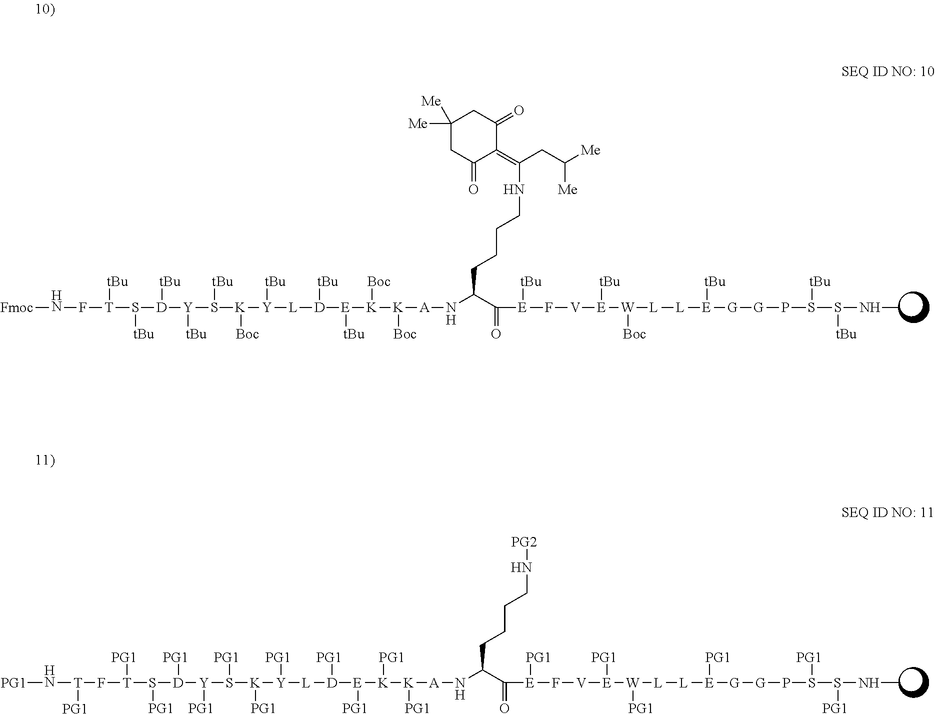

wherein PG1 is a base stable side-chain protecting group,
wherein PG2 is an ivDde, Dde or Alloc side-chain protecting group

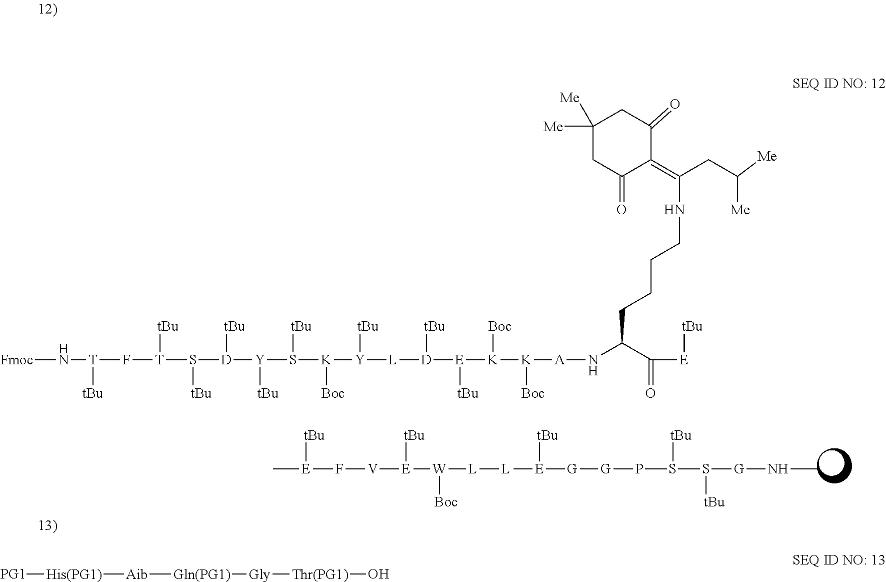

wherein PG1 is a base stable side-chain protecting group

Boc-His(Dnp)-Aib-Gln(Trt)-Gly-Thr(^{t}Bu)-OH 14) SEQ ID NO: 14

PG1-His(PG1)-Aib-Gln(PG1)-Gly-OH 15) SEQ ID NO: 15

wherein PG1 is a base stable side-chain protecting group

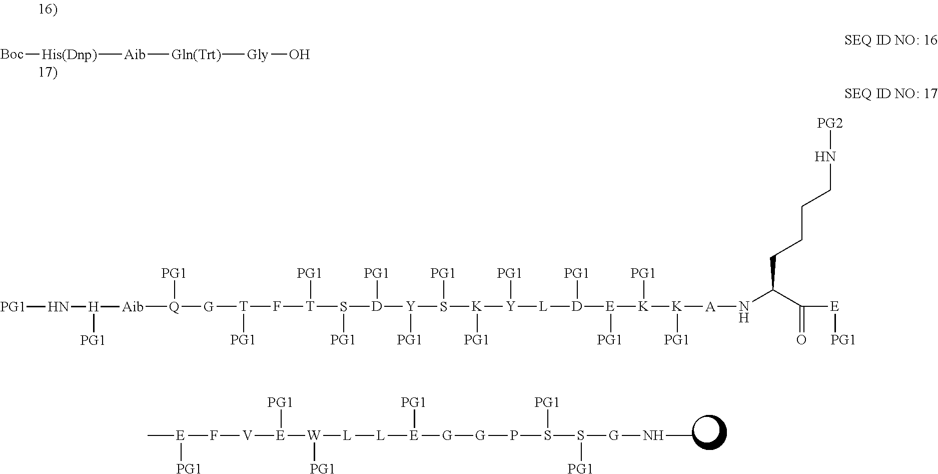

wherein PG1 is a base stable side-chain protecting group,
and wherein PG2 is an ivDde, Dde or Alloc side-chain protecting group

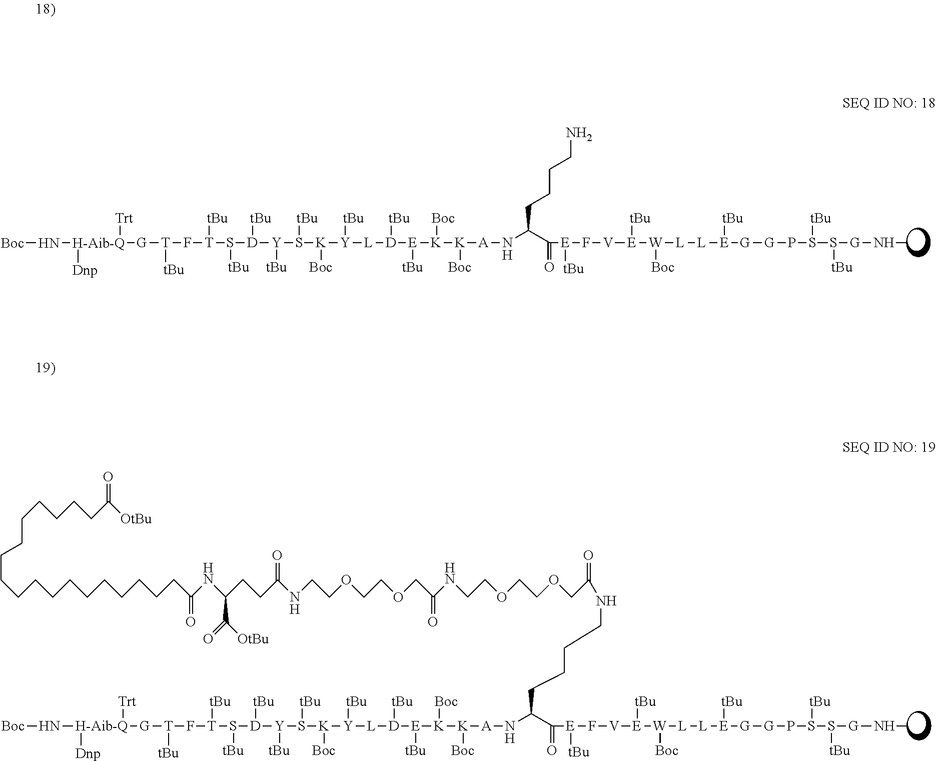

1. A process for the preparation of a compound of the following formula:
H₂N-H-Aib-Q-G-T-F-T-S-D-Y-S-K-Y-L-D-E-K-K-A-K-E-F-V-E-W-L-L-E-G-G-P-S-S-G-NH₂
wherein Lys at position 20 is chemically modified by conjugation of the epsilon-amino group of the Lys side chain with ([2-(2-aminoethoxy)-ethoxy]-acetyl)₂-(γ-Glu)-CO—(CH₂)₁₈CO₂H (SEQ ID NO: 1),
said process comprising the steps of:
(i) solid-phase synthesis of a compound of the following formula 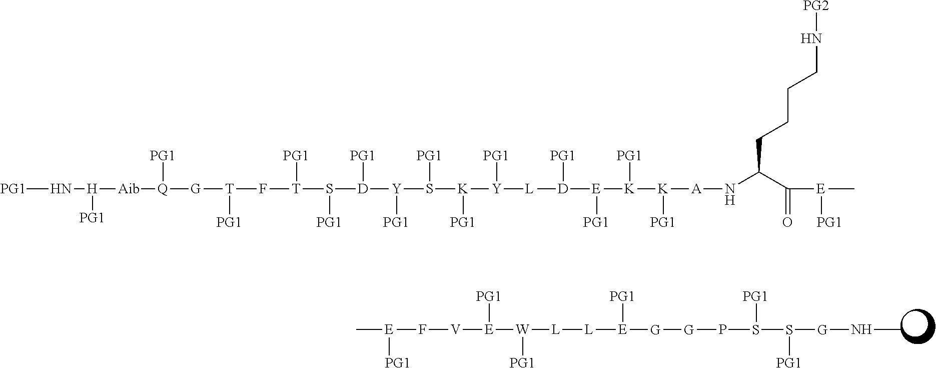
wherein PG1 is a base stable side-chain protecting group,
wherein Thr at position 5 is optionally protected by PG1,
and wherein PG2 is an ivDde, Dde or Alloc side-chain protecting group (SEQ ID NO: 2)
(ii) selectively acylating the compound at the Lys at position 20 (SEQ ID NO: 7) by selectively de-protecting said Lys and coupling the resulting Lys-NH₂ (SEQ ID NO: 5) with ^{t}BuO-C₂₀-γGlu(^{t}Bu)-AEEA-AEEA-OH; and
(iii) cleaving the acylated compound from the solid support and removal of the remaining side chain protecting groups; and
(iv) purifying the compound.

2. A process according to claim 1, wherein PG1 is:
(a) Boc for Trp and Lys;
(b) O^{t}Bu for Asp and Glu;
(c)^{t}Bu for Ser, Thr and Tyr;
(d) Trt for Gln; and
(e) di-Boc for His.

3. A process according to claim 1, wherein PG2 is ivDde or Dde.

4. (canceled)

5. A process according to claim 3, wherein the Lys at position 20 is selectively de-protected by reaction with a solution comprising hydrazine hydrate.

6. A process according to claim 5, wherein the solution comprises 1%-15% w/w hydrazine hydrate in DMF, NMP, NBP or DMSO.

7. A process according to claim 5, wherein the solution comprises 8% w/w hydrazine hydrate in DMF.

8. A process according to claim 1, wherein PG2 is Alloc.

9. A process according to claim 8, wherein the Lys at position 20 is selectively de-protected by reaction with Pd(PPh₃)₄ in the presence of scavengers, preferably H₃N·BH3, Me₂NH·BH3, or PhSiH₃.

10. A process according to claim 1, wherein PG1 is:
(a) Boc for Trp and Lys;
(b) O^{t}Bu for Asp and Glu;
(c)^{t}Bu for Ser, Thr and Tyr;
(d) Trt for Gln; and
(e) di-Boc for His,
wherein PG2 is ivDde,
wherein the solid-phase synthesis of the compound (SEQ ID NO: 3) of step (i) is performed on a Fmoc amide resin solid support and comprises Fmoc deprotection of the amide resin and sequential coupling of the following:
(01) Fmoc-L-Gly-OH;
(02) Fmoc-L-Ser(^{t}Bu)-OH;
(03) Fmoc-L-Ser(^{t}Bu)-OH;
(04) Fmoc-L-Pro-OH;
(05) Fmoc-L-Gly-OH;
(06) Fmoc-L-Gly-OH;
(07) Fmoc-L-Glu(O^{t}Bu)-OH;
(08) Fmoc-L-Leu-OH;
(09) Fmoc-L-Leu-OH;
(10) Fmoc-L-Trp(Boc)-OH;
(11) Fmoc-L-Glu(O^{t}Bu)-OH;
(12) Fmoc-L-Val-OH;
(13) Fmoc-L-Phe-OH;
(14) Fmoc-L-Glu(O^{t}Bu)-OH;
(15) Fmoc-Lys(ivDde)-OH;
(16) Fmoc-L-Ala-OH;
(17) Fmoc-L-Lys(Boc)-OH;
(18) Fmoc-L-Lys(Boc)-OH;
(19) Fmoc-L-Glu(O^{t}Bu)-OH
(20) Fmoc-L-Asp(O^{t}Bu)-OH
(21) Fmoc-L-Leu-OH;
(22) Fmoc-L-Tyr(^{t}Bu)-OH;
(23) Fmoc-L-Lys(Boc)-OH;
(24) Fmoc-L-Ser(^{t}Bu)-OH;
(25) Fmoc-L-Tyr(^{t}Bu)-OH;
(26) Fmoc-L-Asp(O^{t}Bu)-OH;
(27) Fmoc-L-Ser(^{t}Bu)-OH;
(28) Fmoc-L-Thr(^{t}Bu)-OH;
(29) Fmoc-L-Phe-OH;
(30) Fmoc-Gly-Thr(ψ^{Me,Me}Pro)-OH;
(31) Fmoc-L-Gln(Trt)-OH;
(32) Fmoc-Aib-OH; and
(33) Boc-L-His(Boc)-OH.

11. A process according to claim 1, wherein PG1 is:
(a) Boc for Trp and Lys;
(b) O^{t}Bu for Asp and Glu;
(c)^{t}Bu for Ser, Thr and Tyr;
(d) Trt for Gln; and
(e) Boc(Dnp) for His,
wherein PG2 is ivDde,
wherein the solid-phase synthesis of the compound (SEQ ID NO: 4) of step (i) is performed on a Fmoc amide resin solid support and comprises Fmoc deprotection of the amide resin and sequential coupling of the following:
(01) Fmoc-L-Gly-OH;
(02) Fmoc-L-Ser(^{t}Bu)-OH;
(03) Fmoc-L-Ser(^{t}Bu)-OH;
(04) Fmoc-L-Pro-OH;
(05) Fmoc-L-Gly-OH;
(06) Fmoc-L-Gly-OH;
(07) Fmoc-L-Glu(O^{t}Bu)-OH;
(08) Fmoc-L-Leu-OH;
(09) Fmoc-L-Leu-OH;
(10) Fmoc-L-Trp(Boc)-OH;
(11) Fmoc-L-Glu(O^{t}Bu)-OH;
(12) Fmoc-L-Val-OH;
(13) Fmoc-L-Phe-OH;
(14) Fmoc-L-Glu(O^{t}Bu)-OH;
(15) Fmoc-Lys(ivDde)-OH;
(16) Fmoc-L-Ala-OH;
(17) Fmoc-L-Lys(Boc)-OH;
(18) Fmoc-L-Lys(Boc)-OH;
(19) Fmoc-L-Glu(O^{t}Bu)-OH
(20) Fmoc-L-Asp(O^{t}Bu)-OH
(21) Fmoc-L-Leu-OH;
(22) Fmoc-L-Tyr(^{t}Bu)-OH;
(23) Fmoc-L-Lys(Boc)-OH;
(24) Fmoc-L-Ser(^{t}Bu)-OH;
(25) Fmoc-L-Tyr(^{t}Bu)-OH;
(26) Fmoc-L-Asp(O^{t}Bu)-OH;
(27) Fmoc-L-Ser(^{t}Bu)-OH;
(28) Fmoc-L-Thr(^{t}Bu)-OH;
(29) Fmoc-L-Phe-OH;
(30) Boc-His(Dnp)-Aib-Gln(Trt)-Gly-Thr(^{t}Bu)-OH.

12. A process according to claim 1, wherein PG1 is:
(a) Boc for Trp and Lys;
(b) O^{t}Bu for Asp and Glu;
(c)^{t}Bu for Ser, Thr and Tyr;
(d) Trt for Gln; and
(e) Boc(Dnp) for His,
wherein PG2 is ivDde,
wherein the solid-phase synthesis of the compound (SEQ ID NO: 4) of step (i) is performed on a Fmoc amide resin solid support and comprises Fmoc deprotection of the amide resin and sequential coupling of the following:
(01) Fmoc-L-Gly-OH;
(02) Fmoc-L-Ser(^{t}Bu)-OH;
(03) Fmoc-L-Ser(^{t}Bu)-OH;
(04) Fmoc-L-Pro-OH;
(05) Fmoc-L-Gly-OH;
(06) Fmoc-L-Gly-OH;
(07) Fmoc-L-Glu(O^{t}Bu)-OH;
(08) Fmoc-L-Leu-OH;
(09) Fmoc-L-Leu-OH;
(10) Fmoc-L-Trp(Boc)-OH;
(11) Fmoc-L-Glu(O^{t}Bu)-OH;
(12) Fmoc-L-Val-OH;
(13) Fmoc-L-Phe-OH;
(14) Fmoc-L-Glu(O^{t}Bu)-OH;
(15) Fmoc-Lys(ivDde)-OH;
(16) Fmoc-L-Ala-OH;
(17) Fmoc-L-Lys(Boc)-OH;
(18) Fmoc-L-Lys(Boc)-OH;
(19) Fmoc-L-Glu(O^{t}Bu)-OH
(20) Fmoc-L-Asp(O^{t}Bu)-OH
(21) Fmoc-L-Leu-OH;
(22) Fmoc-L-Tyr(^{t}Bu)-OH;
(23) Fmoc-L-Lys(Boc)-OH;
(24) Fmoc-L-Ser(^{t}Bu)-OH;
(25) Fmoc-L-Tyr(^{t}Bu)-OH;
(26) Fmoc-L-Asp(O^{t}Bu)-OH;
(27) Fmoc-L-Ser(^{t}Bu)-OH;
(28) Fmoc-L-Thr(^{t}Bu)-OH;
(29) Fmoc-L-Phe-OH;
(30) Fmoc-L-Thr(^{t}Bu)-OH; and
(31) Boc-His(Dnp)-Aib-Gln(Trt)-Gly-OH.

13. A process according to claim 10, wherein the resin solid support is a Fmoc amide resin solid support and the solid phase synthesis comprises Fmoc deprotection of the resin.

14. A process according to claim 13, wherein the Fmoc amide resin solid support is a Sieber resin.

15. A process according to claim 1, wherein step (iii) further comprises adjusting the pH of a solution comprising the cleaved and deprotected compound to 7.0-8.0, stirring for 1-24 hours, subsequently adjusting the pH of the solution to 1.0-3.0, and stirring for 1-24 hours.

16. A process according to claim 1, wherein the purification of the compound comprises subjecting the compound produced by step (iii) to chromatographic purification.

17. A process according to claim 16, wherein the chromatographic purification is HPLC or reverse phase HPLC.

18. A process according to claim 16 wherein the purification further comprises the steps of (i) adding the chromatographic eluent to a solution comprising aqueous sodium hydroxide or aqueous sodium bicarbonate to form a sodium salt of the compound in solution, (ii) precipitating the sodium salt of the compound from solution and (iii) filtering, washing and drying the precipitated sodium salt of the compound.

19. A process for the preparation of a compound of the following formula: 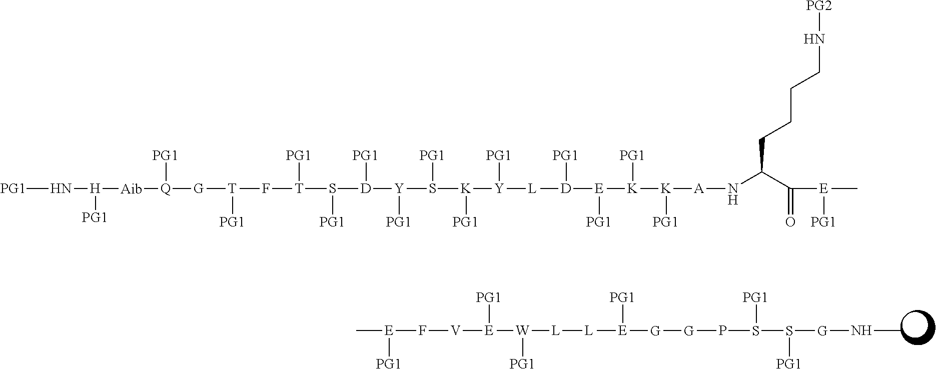
wherein PG1 is a base stable side-chain protecting group,
wherein PG2 is an ivDde, Dde or Alloc side-chain protecting group (SEQ ID NO: 17),
and wherein said process comprises the steps of:
(i) solid-phase synthesis of a compound of the following formula: 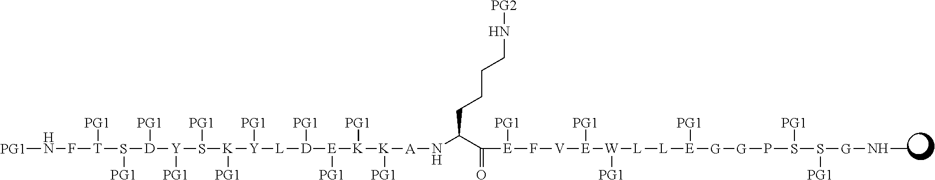
wherein PG1 is a base stable side-chain protecting group,
and wherein PG2 is an ivDde, Dde or Alloc side-chain protecting group (SEQ ID NO: 9); and
(ii) coupling the compound of step (i) with a pentamer of the following formula:
PG1-His(PG1)-Aib-Gln(PG1)-Gly-Thr(PG1)-OH
wherein PG1 is a base stable side-chain protecting group (SEQ ID NO: 13).

20-22. (canceled)

23. A process for the preparation of a compound of the following formula: 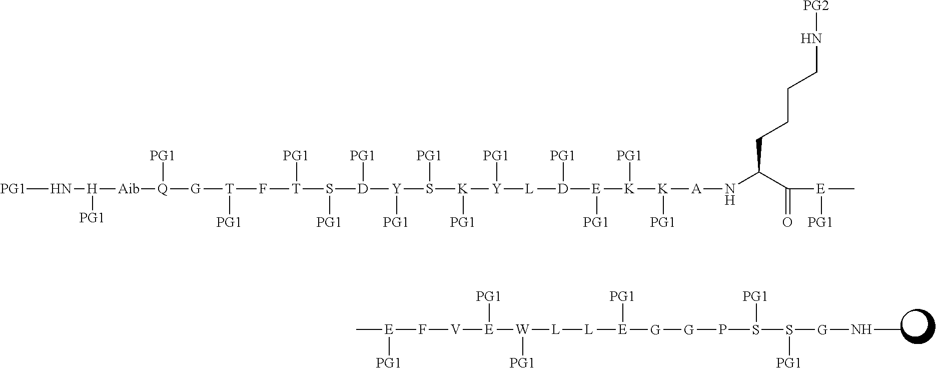
wherein PG1 is a base stable side-chain protecting group,
wherein PG2 is an ivDde, Dde or Alloc side-chain protecting group (SEQ ID NO: 17),
and wherein said process comprises the steps of:
(i) solid-phase synthesis of a compound of the following formula: 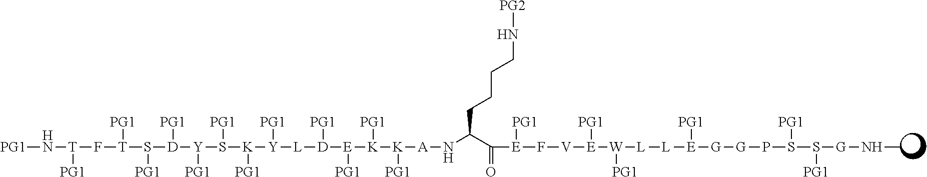
wherein PG1 is a base stable side-chain protecting group,
and wherein PG2 is an ivDde, Dde or Alloc side-chain protecting group (SEQ ID NO: 11); and
(ii) coupling the compound of step (i) with a tetramer of the following formula:
PG1-His(PG1)-Aib-Gln(PG1)-Gly-OH
wherein PG1 is a base stable side-chain protecting group (SEQ ID NO: 15).

24-26. (canceled)

27. A process for the preparation of a sodium salt of the compound of the following formula:
H₂N-H-Aib-Q-G-T-F-T-S-D-Y-S-K-Y-L-D-E-K-K-A-K-E-F-V-E-W-L-L-E-G-G-P-S-S-G-NH₂
wherein lysine (Lys/K) at position 20 is chemically modified by conjugation of the epsilon-amino group of the lysine side chain with ([2-(2-aminoethoxy)-ethoxy]-acetyl)₂-(γ-Glu)-CO—(CH₂)₁₈CO₂H (SEQ ID NO: 1)
said process comprising the steps of:
(i) adding aqueous sodium hydroxide or aqueous sodium bicarbonate to a solution comprising the compound of SEQ ID NO: 1 to form a sodium salt of the compound in solution;
(ii) precipitating the sodium salt of the compound from solution; and
(iii) filtering, washing and drying the precipitated sodium salt of the compound of SEQ ID NO: 1.

28. A compound having a formula selected from the group consisting of SEQ ID NO: 3 SEQ ID NO:4, SEQ ID NO: 10, SEQ ID NO:12, SEQ ID NO:13 and SEQ ID NO:15.

29-35. (canceled)
